(19) United States
(12) Patent Application Publication
(10) Pub. No.: US 2023/0073679 A1
(43) Pub. Date: Mar. 9, 2023

(54) SYSTEM AND METHOD FOR FUNCTIONAL STABILITY MEASUREMENT OF KNEE JOINTS

(71) Applicant: Medfit International AG, Kapfenberg (AT)

(72) Inventors: Thomas Paszicsnyek, Bruck an der Mur (AT); Christian Feigele, Bruck an der Mur (AT)

(21) Appl. No.: 17/701,510

(22) Filed: Mar. 22, 2022

Related U.S. Application Data
(60) Provisional application No. 63/173,626, filed on Apr. 12, 2021.

(51) Int. Cl. A61F 2/46; G16H 20/40; G16H 40/63

(52) U.S. Cl. CPC A61F 2/461; G16H 20/40; G16H 40/63; A61F 2002/4633; A61F 2002/4666

(57) ABSTRACT

A method and system for guiding knee replacement procedures, the method comprising receiving first data values from one or more sensors and one or more functional measurement devices, performing a reference measurement based on the first data values from the one or more sensors and the one or more functional measurement devices prior to a femoral cut, generating joint replacement recommendations based on the reference measurement, receiving second data values from the one or more sensors and the one or more functional measurement devices, performing a control measurement based on the second data values from the one or more sensors and the one or more functional measurement devices after a femoral cut based on the joint replacement recommendations, receiving third data values from the one or more functional measurement devices, and performing a pressure test that measures loading, balance, and kinematic behavior at medial and lateral condyles based on the third data values.

### CROSS REFERENCE TO RELATED APPLICATION

This application claims the priority of U.S. Provisional Application No. 63/173,626, entitled “SYSTEM AND METHOD FOR DYNAMIC LIGAMENT BALANCING FOR IMPLANTING KNEE PROSTHESIS,” filed on Apr. 12, 2021, the disclosure of which is hereby incorporated by reference in its entirety.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

### Field of the Invention

This application generally relates to joint functionality, and in particular, analyzing knee stability geometry and functional ability to aid in knee replacement procedures.

### Description of the Related Art

Joint replacement surgeries have been performed for all major and some minor joints for decades. In the beginning, only the replacement of the surface was done and little to no attention was paid to the surrounding functional structures. Within the last decade, more and more surgeons have recognized that the collaboration between these structures is key to successful joint replacement. The functionality of joints is based on different support components: bone with articular surfaces covered with cartilage, capsule, ligaments and muscle envelope. Together, these components provide proper functionality of a joint. This condition is valid on all major and minor joints, only the importance of specific components are different. By example, for the function of a knee, the primary stabilizing system is ligaments, secondarily supported by the muscle envelope.

Several devices have been developed to assist surgeons in proper placement of joint implants, but only a few tools exist that assess functional behavior of the joint implants. One existing system that evaluates joint implant functional behavior is the “Optimized Positioning System” by Corin, which calculates the correct implant position based on dynamic x-rays. However, such a system is an indirect way to assess functionality. There is thus a need for a system that evaluates the leading parts of joint stability and guides surgeons on how to replace the joint surfaces/joint during orthopedic surgical procedures.

### SUMMARY OF THE INVENTION

The present invention provides a method and system for guiding knee replacement procedures. According to one embodiment, the method comprises receiving first data values from one or more sensors and one or more functional measurement devices, performing a reference measurement based on the first data values from the one or more sensors and the one or more functional measurement devices prior to a femoral cut, rendering the reference measurement on a client device, generating joint replacement recommendations based on the reference measurement, receiving second data values from the one or more sensors and the one or more functional measurement devices, performing a control measurement based on the second data values from the one or more sensors and the one or more functional measurement devices after a femoral cut based on the joint replacement recommendations, rendering the control measurement on the client device, receiving third data values from the one or more functional measurement devices, performing a pressure test that measures loading, balance, load distribution, and kinematic movements at medial and lateral condyles based on the third data values, and rendering the pressure test on the client device.

A pairing signal broadcasted from the one or more sensors may be detected and a connection with the one or more sensors may be established. The one or more sensors may be calibrated based on movement of a joint associated with the one or more sensors, the movement comprising at least one of moving forward and backward, flexing and extending the joint attached to the one or more sensors, and rotating the joint. A pairing signal broadcasted from the one or more functional measurement devices may be detected and a connection with the one or more sensors may be established. The one or more functional measurement devices may be switched on by physically depressing and holding one or more paddles on the functional measurement device. Calibrating the one or more functional measurement devices may comprise releasing the one or more paddles after the depressing.

The one or more functional measurement devices may include one or more paddles that measure distance traveled by the paddle surfaces when compressed between a tibial surface and the medial and lateral condyles. Performing the reference measurement may comprise recording reference data values from the one or more sensors and the functional measurement devices at given angles. The reference data values may include paddle heights at the medial and lateral condyles along with corresponding pressures. The joint replacement recommendations may include determining and/or calculating one or more of ideal joint planes, alignment, cutting planes, cutting thickness, distal femur angle, femoral rotation, implant types and sizes. Performing the control measurement may further comprise recording control data values from the one or more sensors and the functional measurement devices at given angles. The control data values may include paddle heights at the medial and lateral condyles along with corresponding pressures. Tension may be displayed based on the control data values. A further measurement may be performed to determine the pressures.

According to one embodiment, the system comprises a processor and a memory having executable instructions stored thereon that when executed by the processor cause the processor to receive first data values from one or more sensors and one or more functional measurement devices, perform a reference measurement based on the first data values from the one or more sensors and the one or more functional measurement devices prior to a femoral cut, render the reference measurement on a user interface, generate joint replacement recommendations based on the reference measurement, receive second data values from the one or more sensors and the one or more functional measurement devices, perform a control measurement based on the second data values from the one or more sensors and the one or more functional measurement devices after a femoral cut based on the joint replacement recommendations, render the control measurement on the user interface, receive third data values from the one or more functional measurement devices, perform a pressure test that measures loading, balance, and kinematic behavior at the medial and lateral condyles through a range of motion based on the third data values, and render the pressure test on the user interface.

The processor may be further configured to detect a pairing signal broadcasted from the one or more sensors; and establish a connection with the one or more sensors. The processor may be further configured to calibrate the one or more sensors based on movement of a joint associated with the one or more sensors, the movement comprising at least one of moving forward and backward, flexing and extending the joint attached to the one or more sensors, and rotating the joint. The processor may be configured to detect a pairing signal broadcasted from the one or more functional measurement devices and establish a connection with the one or more sensors. The one or more functional measurement devices may be switched on by physically depressing and holding one or more paddles on the functional measurement device. The one or more functional measurement devices may be calibrated by releasing the one or more paddles after the depressing. The one or more functional measurement devices may include one or more paddles that measure distance traveled by the paddle surfaces when compressed between a tibial surface and the medial and lateral condyles. The joint replacement recommendations may include determining and/or calculating one or more of ideal joint planes, alignment, cutting planes, cutting thickness, distal femur angle, femoral rotation, implant types and sizes.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 illustrates a computing system according to an embodiment of the present invention.

FIG. 2 illustrates a perspective view of the front, left, and top sides of a ligament balancing tool for unicondylar knee arthroplasty according to an embodiment of the present invention.

FIG. 3 illustrates a perspective view of the front, left, and top sides of a ligament balancing tool for bicondylar knee arthroplasty according to an embodiment of the present invention.

FIG. 4 illustrates an exemplary sensor unit configured on a femoral surface according to an embodiment of the present invention.

FIGS. 5A, 5B, and 6 illustrate a patella balancing device for patella replacement according to an embodiment of the present invention.

FIGS. 7 and 8 illustrate exemplary interfaces for patella replacement according to an embodiment of the present invention.

FIGS. 9 and 10 illustrate a femoral balancing device for femur replacement according to an embodiment of the present invention.

FIG. 11 illustrates an exemplary interface for femur replacement according to an embodiment of the present invention.

FIG. 12 illustrates patellofemoral balancing devices for patella and femur replacement according to an embodiment of the present invention.

FIG. 13 illustrates an exemplary interface for patella and femur replacement according to an embodiment of the present invention.

FIG. 14 illustrates a flowchart of a method for guiding knee replacement procedures according to an embodiment of the present invention.

FIGS. 15 through 45 illustrate exemplary user interfaces of a functional stability measurement and analysis system according to an embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

The present application discloses a system for assessing joint stability and replicating the stability in joint replacement surgery. The disclosed system combines proper implant position and functional matching to achieve joint replacements that are satisfactory in performance. The disclosed system may comprise a computing system that acquires joint tensioning data to perform objective adjustment of the dynamic soft tissue tension in the leg of patients during the course of the implantation of an artificial knee joint in accordance with individual capabilities and joint function.

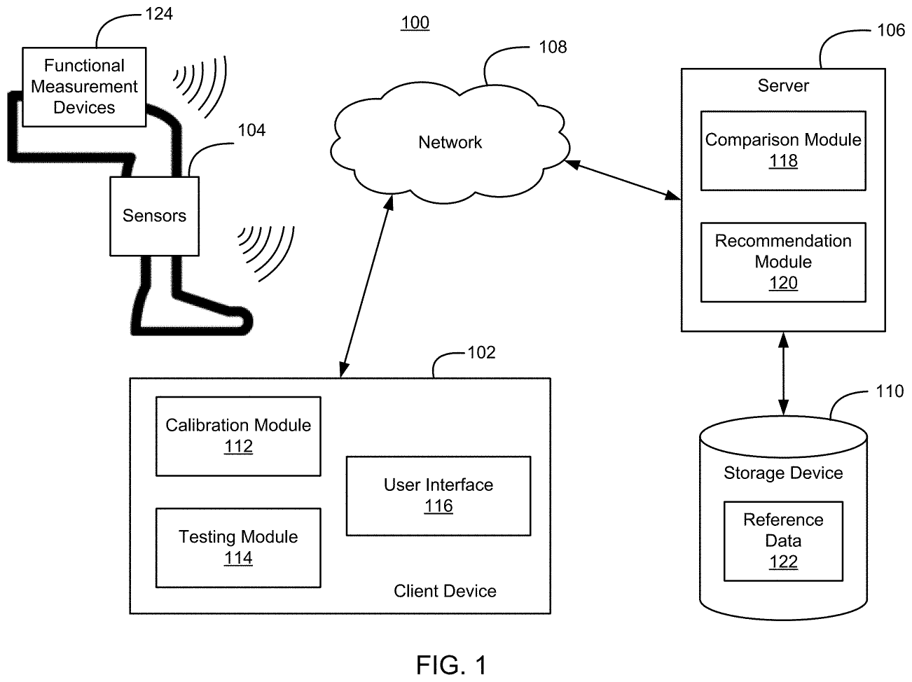

FIG. 1 presents a computing system according to an embodiment of the present invention. The system 100 presented in FIG. 1 includes client device 102, sensors 104, functional measurement devices 124, server 106, network 108, and storage device 110. Client device 102 may comprise a computing device (e.g., desktop computer, television device, laptop, personal digital assistant (PDA), smartphone, tablet computer, e-book reader, smart watch and smart wearable devices, or any computing device having a central processing unit and memory unit capable of connecting to a network). The client device 102 may also comprise a graphical user interface (GUI) or a browser application provided on a display (e.g., monitor screen, LCD or LED display, projector, etc.). Client device 102 may include or execute a variety of operating systems, including a personal computer operating system, such as a Windows, Mac OS or Linux, or a mobile operating system, such as iOS, Android, or Windows Phone, or the like. The client device 102 may include or may execute a variety of possible applications, such as a client software application enabling communication with other devices.

Sensors 104 may comprise gyro sensor units that each include movement tracking, power, circuitry, and wireless communication components. The sensors 104 may detect rotational motion and changes in orientation, and communicate that information to the system 100 for tracking the 3D position of the sensors 104 in three dimensions. The sensors 104 may be for single-use only and each have a defined size and shape to fit certain dimensions (e.g., 20×20×8 mm). The sensors 104 preferably include pins or other anchoring devices for attaching the individual sensor to the bones of the body part being operated on. Each of sensors 104 may be activated by, e.g., a magnetic or manual switch, and identified or labeled according to their position relative to a plurality of predefined positions, and registered with an interface on client device 102.

The sensors 104 in communication with client device 102 may, once activated, form a three-dimensional matrix or grid reference system such that an orientation of, for example, the tibial plane and the femoral joint surface can be modeled and validated with the system 100. The sensors 104 may further include RFID sensors, a control unit, and a central processing unit for pre-calculating data values based on the electrical signals and data from the gyroscopic and RFID sensors. The pre-calculated data values may be sent by the sensors 104 to an application on client device 102 where the data values may be combined with x-rays and evaluation tables including patient information.

Functional measurement devices 124 may be inserted at a knee joint where a medial condyle and a lateral condyle of a femur are placed on respective paddle surfaces and a bottom surface of a base of the one or more functional measurement devices 124 may rest above a tibial surface. The functional measurement devices 124 may measure distance traveled (or displacement) by the paddle surfaces when compressed by the medial condyle and the lateral condyle of the femur bone against the tibia. The measured distance traveled by the paddle surfaces may be used to calculate forces or tension at the medial or lateral condyles of a knee. Functional measurement devices may comprise ligament balancing tools which are described in further detail below.

The sensors 104 and functional measurement devices 124 may be individually activated to send active signals including location and measurement data to client device 102 either over network 108 or directly via a wireless communication connection, such as by Bluetooth, Wi-Fi, or near-field communication (NFC). Data values received by the client device 102 may be used to measure various parameters of a knee to generate recommendation for planning of a knee replacement procedure, as well as guide the surgeon during the knee replacement procedure.

Measurement procedures may be conducted (pre-, intra-, and post-operative) over an entire range of motion of the knee using the sensors 104 and functional measurement devices 124, where critical values, such as measurements at 0°, 30°, 60°, and 90° of flexion, may be rendered on a graphic user interface of a dedicated application. The critical values may be customizable to measurements of any predetermined angles of flexion. A surgical procedure may start with a preparation of the soft tissue by removing possible osteophytes and cutting the medial or lateral part of the tibial plateau. After doing so, the functional measurement devices 124 may be mounted with a pin to secure a correct position between the femur and the tibia. Medial or lateral forces or load data (represented by displacement) may be captured using the functional measurement devices 124 and transferred to a computing device to create a representative tension profile of the knee during 0° to 90° flexion of the knee.

Client device 102 may include a calibration module 112, testing module 114, and user interface 116. Calibration module 112 may include logic for executing calibration procedures with the sensors 104 by instructing a user through user interface 116 to perform several operations such as, moving forward and backward, flexing and extending the knee joint attached to the sensor, or rotating the knee joint. The calibration module 112 may guide positioning of the sensors 104 to ensure consistent measurement results that can be compared with data from reference data 122.

Testing module 114 may include software or programming including testing procedures for measuring or evaluating the joint measured by the sensors 104 and functional measurement devices 124, which may collectively represent functional stability of the joint. According to one embodiment, the system may further include additional equipment to gather information such as, gait analysis, load distribution analysis, and motion analysis. Exemplary equipment may include special cameras and insoles employed to gather the information. For example, gait analysis, load distribution analysis, and motion analysis may include monitoring data from insoles worn by an individual and capturing movement during a walking procedure, treadmills, and load platforms.

Reference measurement data may be gathered by testing module 114 to evaluate the function of an affected joint. The reference measurement data may comprise a recording of data values received from the sensors 104. The client device may compare the reference measurement data with reference data 122 by transmitting the reference measurement data to a comparison module 118 at server 106. Comparison module 118 may comprise artificial intelligence or computing logic configured to compare the reference measurement data with reference data 122 stored in storage device 110.

Reference data 122 may be accessed by client device 102 through server 106 over network 108, for example, as a cloud-based service, or a service subscription. The reference data 122 may comprise data of healthy people with normal joint function that can be compared with the data values from the sensors 104. Comparing the reference measurement data with the reference data 122 may further include determining a control cohort divided in different age groups and formulating an algorithm for calculating average values of joint parameters in the patient's age. Results of the comparison made by comparison module 118 may be transmitted to the client device 102. The reference data may also include preoperative and postoperative data of other patients.

The client device 102 may further generate recommendations based on the comparison by referencing recommendation module 120. The recommendation module 120 may generate joint replacement options by determining and/or calculating one or more of ideal joint planes, alignment, cutting planes, implant types and sizes that are individualized to the patient based on the comparison with reference data 122 (e.g., the average values in the patient's age) to regain full functionality and range of motion. Deformities and pain restricted movement factors may also be provided by the client device 102 to be considered in generating the joint replacement options by the recommendation module 120.

The recommendations may be transmitted to client device 102 based on the results of the comparison from comparison module 118. The recommendations may include a preoperative view based on the reference measurement data, comparison, and optionally other preoperative data. The reference measurement data and recommendations may be rendered by the client device 102 on the user interface 116.

Data values and recommendations may be transmitted to a three-dimensional orientation system for validating intra-operative procedure and correct positioning by a surgeon. Further details of the three-dimensional orientation system is described in commonly owned U.S. patent application Ser. No. 16/904,823, entitled “THREE-DIMENSIONAL ORIENTATION SYSTEM AND METHOD FOR ORTHOPEDIC SURGERY,” filed on Jun. 18, 2021, which is herein incorporated by reference in its entirety. Alternatively, if a three-dimensional orientation system is not used, values for a cutting plane may be determined and displayed together with the recommended implants including sizes and diameters.

Using results from the reference measurement, a femoral cutting block can be adjusted to the correct position and the femoral cuts may be performed. After cutting the femur, a control measurement may also be performed by the testing module 114 using the interface and software application executing on the computing device to verify results. Pre-, intra- and postoperative measurement data (e.g., reference measurement data, control measurement data, etc.) may be stored by the computing device to a cloud database that is secured by an access hierarchy in accordance with data protection regulations. The disclosed system may further identify an individual kinematic situation of a specific patient including the knee motion and range of motion as well as individual limitations and abilities that can affect the ideal configuration of components of the knee replacement.

Server 106, as described herein, may vary widely in configuration or capabilities but is comprised of at least a special-purpose digital computing device including at least one or more central processing units and memory. The server 106 may also include one or more of mass storage devices, power supplies, wired or wireless network interfaces, input/output interfaces, and operating systems, such as Windows Server, Mac OS X, Unix, Linux, FreeBSD, or the like. In an example embodiment, server 106 may include or have access to memory for storing instructions or applications for the performance of various functions and a corresponding processor for executing stored instructions or applications. For example, the memory may store an instance of the server 106 configured to operate in accordance with the disclosed embodiments.

Network 108 may be any suitable type of network allowing transport of data communications across thereof. The network 108 may couple devices so that communications may be exchanged, such as between servers and client devices or other types of devices, including between wireless devices coupled via a wireless network, for example. Network 108 may also include mass storage, such as network attached storage (NAS), a storage area network (SAN), cloud computing and storage, or other forms of computer or machine readable media, for example. In one embodiment, the network may be the Internet, following known Internet protocols for data communication, or any other communication network, e.g., any local area network (LAN) or wide area network (WAN) connection, cellular network, wire-line type connections, wireless type connections, or any combination thereof. Communications and content stored and/or transmitted to and from client device 102 may be encrypted using, for example, the Advanced Encryption Standard (AES) with a 128, 192, or 256-bit key size, or any other encryption standard known in the art.

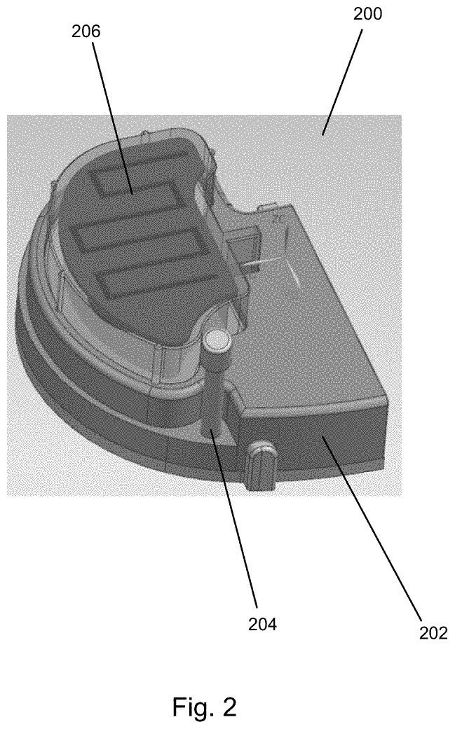

FIG. 2 presents a front perspective view of a ligament balancing tool for unicondylar knee arthroplasty according to an embodiment of the present invention. The ligament balancing tool 200 comprises a base 202 including an integrated sensor. Ligament balancing tool 200 may be singularly inserted at individual compartments of a knee joint where either a medial condyle or a lateral condyle of a femur is placed on a surface of paddle 206 and a bottom surface of base 202 may rest above a tibia. The integrated sensor may be configured below a paddle 206 to measure distance traveled (or displacement) by paddle 206 when compressed by a medial condyle or the lateral condyle when ligament balancing tool 200 is placed between the tibia and femur bone. The measured distance traveled by paddle 206 may be used to calculate forces at the medial or lateral condyles of a knee. That is, the ligament balancing tool 200 may be used to measure medial forces of a right knee or lateral forces on a left knee. Conversely, a similar but inverted or mirror-version of ligament balancing tool 200 may be used to measure medial forces of a left knee or lateral forces on a right knee. Base 202 may be produced in different sizes in accordance to variation in sizes of the human knee. According to one embodiment, the ligament balancing tool 200 may be produced as a sterile packaged single-use part and combined into a product set. Additionally, the ligament balancing tool 200 may be either platform-dependent (e.g., designed for specific products from different manufacturers) or platform independent (universally compatible, e.g., via an adapter).

Paddle 206 is supported on base 202 in a static position (when the paddle 206 is unloaded) by a support structure coupled to an underlying spring coil and a torsion coil having a given resistance. The spring coil and torsion coil is attached to base 202 and connects the integrated sensor with the paddle 206.

The paddle 206 may be depressed by applying pressure that causes the paddle 206 to move from a static (or fully upright) position to a depressed (or loaded) position. The spring coil and the torsion coil under the paddle 206 provide resistance on the paddle 206 over a lifting distance range, for example, between 8 mm (fully depressed) to 22 mm (fully upright) from the base 202. However, practical application of the ligament balancing tool 200 may only require an operational lifting distance range between 8.5 mm to 14.5 mm. Pressure applied on the paddle 206 is transferred to the integrated sensor by the spring and torsion coils. Resistance provided by the spring and torsion coil may be either measured by the integrated sensor or determined using a known distance to resistance ratio such that integrated sensor and/or a computing device communicatively coupled to the integrated sensor may calculate an amount of tension required to displace or move paddle 206 a distance measured by the integrated sensor. According to one embodiment, additional sensors may also be embedded in the paddle 206, or the spring/torsion coils.

Integrated sensor is comprised of force and/or distance measurement devices such as piezo or force-sensitive resistor (FSR) sensors that are commercially available. However, capacitive sensors and other strain gauges may also be used accordingly to their durability and reliability. The integrated sensor may create voltages or signals representative of the amount of force or tension produced on the paddle 206. The integrated sensor may be connected to an electrical or signal bus comprised in a connecting cable. The connecting cable may be adapted from base 202 to a connector (e.g., via a wired connection) for communication with external electronics that are able to receive and convert the voltages or signals from integrated sensor into data for display and recording. Alternatively, the voltages or signals may be communicated to the external electronics using a wireless signal connection, such as Bluetooth (e.g., version 1.0 through 5.2 or any future versions).

According to one embodiment, the voltages or signals from the integrated sensor may be transmitted to a computing device. The computing device may include software for signal acquisition and processing of the voltages or signals from the integrated sensor to provide a visualization of the measured data. The computing device may comprise computing devices (e.g., desktop computers, terminals, laptops, personal digital assistants (PDA), cell phones, smartphones, tablet computers, or any computing device having a central processing unit and memory unit capable of connecting to a network). The computing device may also comprise a graphical user interface (GUI) or a browser application provided on a display (e.g., monitor screen, LCD or LED display, projector, etc.).

The ligament balancing tool 200 may be placed between the femur and the tibia such that the paddle 206 is beneath the femur (or femoral component) and base 202 is above the tibia (or tibial baseplate). Paddle 206 may further include indentation. Indentation may be provided to accommodate the femur bone, specifically the medial condyle or the lateral condyle. Accordingly, the femur bone is able to fit in indentation without slippage when pressed against paddle 206.

The ligament balancing tool 200 may be used in a unicondylar knee arthroplasty procedure. Particularly, a measurement procedure may be conducted (pre-, intra-, and post-operative) over an entire range of motion of the knee using the ligament balancing tool 200, where critical values, such as measurements at 30°, 60°, and 90° of flexion, may be rendered on a graphic user interface of a dedicated application. The critical values may be customizable to measurements of any predetermined angles of flexion. A surgical procedure may start with a preparation of the soft tissue by removing possible osteophytes and cutting the medial or lateral part of the tibial plateau. After doing so, the ligament balancing tool 200 may be mounted with a pin 204 to secure a correct position between the femur and the tibia.

Medial or lateral forces or load data (represented by displacement) may be captured using the ligament balancing tool 200 (via the integrated sensor) and transferred to a computing device to create a representative tension profile of the knee during 0° to 90° flexion of the knee.

Distance values representative of tension measured at the medial collateral ligament by the integrated sensor may be presented along with critical flexion angles on a user interface. A current flexion may be indicated along with an avatar which depicts a virtual representation of the knee at a current flexion angle and the position of the ligament balancing tool on the medial condyle or the lateral condyle. Expected cutting thickness of the medial condyle or the lateral condyle may also be computed and configured on the user interface.

Using the information provided on the user interface as a guide, a femoral cutting block can be adjusted to secure proper stability and function of an implant and also to ensure stability of the medial or lateral collateral ligament. After inserting a femoral trial implant, a control measurement can be performed using the dynamic ligament balancing tool, this time on a tibial baseplate. Performing the control measurement can secure correct and symmetric stability. Additional changes can be made regarding slope and distance until correct values are reached or are in an acceptable range.

A recommended size of implant may be calculated by the disclosed computing device by gathering control data including measurements of tension at the knee. Based on the measurement, the computing device may determine an appropriately sized implant inlay that targets correct tension values over an entire range of motion of the knee. For example, distance values representative of tension measured at the medial collateral ligament by the integrated sensor may be presented at various critical flexion angles on a user interface. A current flexion may be indicated along with an avatar which depicts a virtual representation of the knee with a femoral trial implant at a current flexion angle and the position of the ligament balancing tool on the medial condyle.

The interface may display a distance of, for example, 9 mm starting at 0° flexion. The 9 mm distance at 0° flexion may be a measured distance at the current flexion. As the knee is moved from 0° to 20° flexion, the user interface may generate a notification that indicates a warning and an indication of an issue (that the extension gap is tight). For example, “extension gap is tight,” may indicate that a measured distance at 20° flexion is larger than the distance at 0°. The avatar and notification may be rendered and presented in various colors to indicate issue severity (e.g., yellow for warnings and red for critical faults).

Another notification including a “mismatch between extension and flexion” and “too tight” may indicate that measured distances at certain angle flexions are out-of-range and too small compared to the distances measured at other angle flexions. A recommended inlay thickness may be calculated based on the control data to replace the trial prosthesis with a final prosthesis that can provide appropriate medial distances throughout the entire range of motion (e.g., from 0° to 90°). Distance values may be updated to present distance values calculated by the computing device that are expected from using the recommended inlay thickness.

Pre-, intra- and postoperative measurement data (e.g., reference data, control data, etc.) may be stored by the computing device to a cloud database that is secured by an access hierarchy in accordance with data protection regulations. The disclosed system may identify an individual kinematic situation of a specific patient including the knee motion and range of motion as well as individual limitations and abilities. The pre-, intra- and postoperative measurements can be used for quality control and can be connected to a muscle activity controlled physiotherapy (“MacP”) system (as discussed in commonly-owned U.S. patent application Ser. No. 17/482,028, entitled “SYSTEM AND METHOD FOR AIDING IN PHYSIOTHERAPY BY MEASURING MUSCLE ACTIVITY USING WIRELESS SENSORS,” filed on Sep. 22, 2021, which is incorporated herein by reference) to provide additional information to the individual condition of the patient which can affect the ideal configuration of components of the knee replacement.

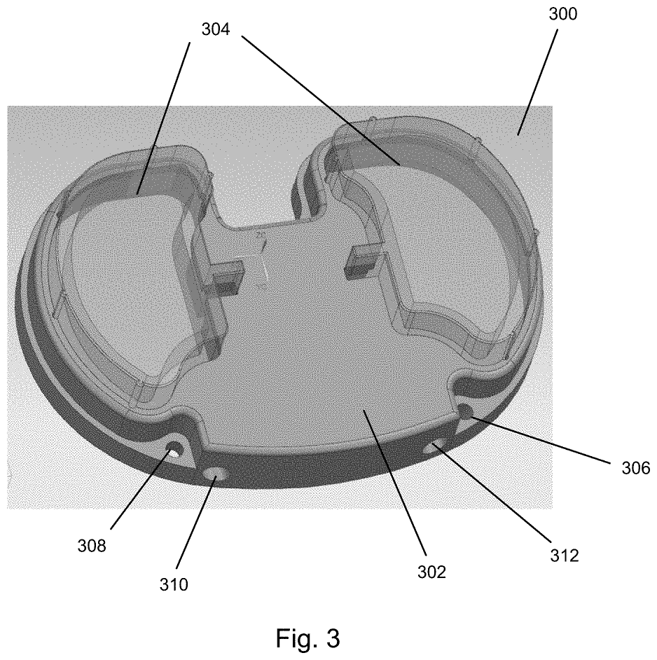

FIG. 3 presents a perspective view and a side view, respectively of a ligament balancing tool for bicondylar knee arthroplasty according to an embodiment of the present invention. The ligament balancing tool 300 comprises a base 302, and paddles 304. Ligament balancing tool 300 may be inserted in a knee joint where a medial condyle and a lateral condyle of a femur are placed on surfaces of the paddles 304 and a bottom surface of base 302 may rest on a surface of a tibia. Paddles 304 are supported on base 302 via a support structure comprising a spring coil system having given resistances to enable an underlying support structure to hold the paddles 304 in a static (or fully upright) position when the paddles 304 are unloaded. The support structures may be coupled to any number (e.g., one or more) of spring coils.

The spring coils may collectively produce a constant tension over an entire lifting distance range of paddles 304. That is, different resistances provided by the spring coils can be combined to produce a configurable static resistance (e.g., 20 newtons, 25 newtons, or 30 newtons). The ligament balancing tool 300 may further include a resistance adjuster that controls variance of resistance provided by the spring coils (e.g., settings of 20 newtons, 25 newtons, and 30 newtons). Vertical spring coils may be positioned vertically under base 302 such that they are substantially perpendicular to overlaying paddles 304, respectively, as well as substantially perpendicular to base 302. Horizontal spring coils may be embedded within respective cavities of base 302 in a horizontal position substantially parallel to paddles 304 as well as substantially parallel to base 302. The vertical and horizontal spring coils may operate in concert with each other to provide given resistances on the paddles 304 over a lifting distance range, for example, between 8 mm (fully depressed) to 22 mm (fully upright) from the base 302. Practical application of the ligament balancing tool 300 may only require an operational lifting distance range between 8.5 mm to 14.5 mm, however, wider distances may also be achieved by additional adapter plates fixed on the base 302. Resistance provided to the paddles 304 may be adjusted by controlling the spring coils via a tensioning system using the resistance adjuster, or alternatively a digital interface on a computing device.

Ligament balancing tool 300 further includes locking apertures 310 and 312 for insertion of locking keys to set the paddles 304 at a static height. For example, paddles 304 may be depressed to given heights and locked in their current positions via the locking apertures 310 and 312. Locking the paddles 304 at a given position may comprise configuring a high or maximum amount of resistance to support the paddles at a given height and allowing for depression of the paddles 304 upon overcoming the configured resistance.

Base 302 may be produced in different sizes in accordance to variation in sizes of the human knee. The base 302 further includes pin apertures 306 and 308 for accepting pins for securing ligament balancing tool 300 to bone, e.g., a tibial surface. A pin positioning block may also be attachable to the ligament balancing tool 300 for providing a guide for pin positioning/drilling to assist a surgeon to perform steps required to prepare the femur and tibia for receiving the implant. Functionality of the pin positioning block is discussed in further detail in U.S. Pat. No. 10,722,168, entitled “DYNAMIC LIGAMENT BALANCING SYSTEM WITH PIN POSITIONING BLOCK,” which is incorporated herein by reference. According to one embodiment, the ligament balancing tool 300 may be produced as a sterile packaged single-use part and combined into a product set. Additionally, the ligament balancing tool 300 may be either platform-dependent (e.g., designed for specific products from different manufacturers) or platform independent (universally compatible, e.g., via an adapter).

Total resistance on the paddles 304 can be adjusted to match intra-operative requirements of a selected implant. For example, cruciate retaining implants may require lower tension than posterior stabilized implants. Horizontal spring coils can be switched on and off (selectively activated) in concert with a vertical spring coil to provide variable resistance via a tensioning system that is embedded within base 302. A vertical spring coil alone may provide a given resistance, such as up to 20 newtons. Horizontal spring coils may each provide additional resistance of, for example, up to 5 newtons to the 20 newtons already provided by the vertical spring coil. That is, one of horizontal spring coils may be selectively activated to provide a total resistance of up to 25 newtons on paddles 304, while activating both horizontal spring coils may provide a total resistance of up to 30 newtons on paddles 304.

Paddles 304 may be configured with one or more sensors that are used to measure medial and lateral forces at the medial and lateral condyles of a knee over an entire range of motion of the knee when the ligament balancing tool 300 is placed between the tibia and femur bone. Pressure may be applied to the paddles 304 to depress the paddles and move them from the static (or fully upright) position to a depressed (or loaded) position. An integrated sensor (not illustrated) may measure an amount of displacement or distance that paddles 304 travel. The amount of displacement or distance may be used to determine the medial and lateral forces. The integrated sensor may be embedded in the paddles 304, spring coils, or support structures within base 302. The integrated sensor may comprise distance and positioning measurement devices, such as a hall sensor. Resistance provided by the spring coils may either measured by the integrated sensor or determined using a known distance to resistance ratio such that the integrated sensor and/or a computing device communicatively coupled to the integrated sensor may calculate an amount of tension required to displace or move the paddles 304 a distance measured by the integrated sensor.

The integrated sensor may create voltages or signals representative of the amount of tension produced on the paddles 304. The integrated sensor may be connected to an electrical or signal bus comprised in a connecting cable. The connecting cable may be adapted from base 302 to a connector (e.g., via a wired connection) for communication with external electronics that are able to receive and convert the voltages or signals from integrated sensor into data for display and recording. Alternatively, the voltages or signals may be communicated to the external electronics using a wireless signal connection, such as Bluetooth (e.g., version 1.0 through 5.2 or any future versions). According to one embodiment, the voltages or signals from the integrated sensor may be transmitted to a computing device. The computing device may include software for signal acquisition and processing of the voltages or signals from the integrated sensor to provide a visualization of the measured data. Medial and lateral forces or load data (e.g., represented by displacement) may be captured using the ligament balancing tool 300 (via sensors) and transferred to a computing device to create a representative tension profile of the knee during 0° to 90° flexion of the knee.

According to another embodiment, additional sensors and transmitters may also be embedded in the ligament balancing tool 300, such as a combination of a gyro sensor, a radio-frequency identification chip, and/or a plane-sensor. This combination enables the ligament balancing tool 300 to transmit data about the position of the ligament balancing tool within a matrix to a computing device which may be used to render an information and graphical aid or guide. Specifically, distance, intercondylar angle (joint angle), tension, and a rotational ratio between the tibia and femur may be calculated and presented by the computing device by using the combination of sensors. The disclosed dynamic ligament balancing tool may be used in conjunction with a three-dimensional orientation system that determines and guides users with respect to correct implant positioning using individual patient kinematics data. The three-dimensional orientation system may include one or more positioning sensors that can be placed on pre-defined anatomical landmarks in a body area to be operated on, and one or more plane-marker sensors for fixing an adjustable cutting block relative to the joint bone being cut, preferably adjustable in three dimensions, in a desired position.

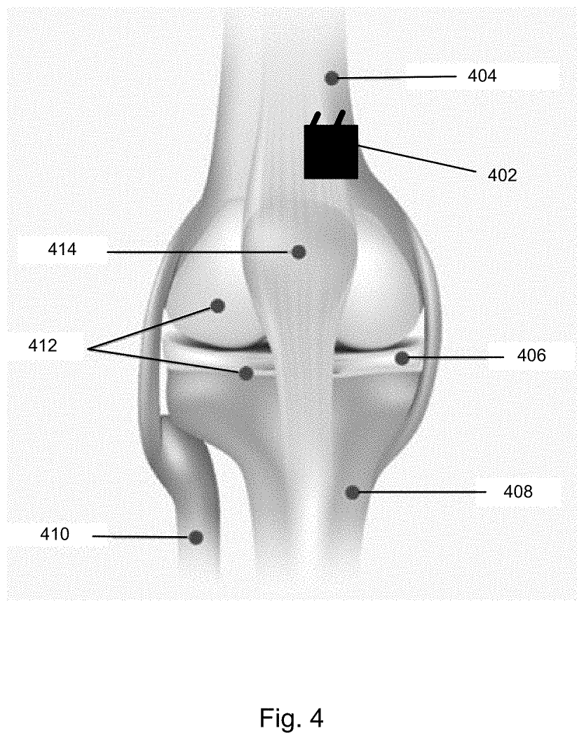

FIG. 4 presents an exemplary sensor unit configured on a femoral surface according to an embodiment of the present invention. The knee region is comprised of a femoral bone 404, meniscus 406, tibia 408, fibula 410, cartilage 412, and patella 414. A sensor 402 is fixed on the anterior cortex of the femoral bone 404. To place the sensor 402, the patella 414 may be moved to the lateral side and can be brought in the normal position after fixing the sensor 402. The sensor 402 may be optical, non-optical, or a combination thereof. Optical sensors may include markers that are observed by system cameras, for example, to determine the position of the markers in a three dimensional space. Non-optical systems may include inertial units (referred to herein generally as “gyro” units), which may include at least one of a gyroscope, magnetometer, an accelerometer, a RFID chip, or a combination thereof, to measure the position and/or orientation of each of the sensor units in three dimensions. Similarly, magnetic systems may be used to calculate position and/or orientation of the sensor 402 based on the relative magnetic flux of the sensor 402 to three orthogonal coils.

Sensor 402 according to at least one embodiment includes a gyro sensor unit that includes movement tracking, power, circuitry, and wireless communication components. The gyro sensor unit may detect rotational motion and changes in orientation, and communicate that information to a computing device for tracking the three-dimensional position of the sensor in three dimensions. The sensor 402 preferably includes pins or other anchoring devices for attaching the individual sensor to the femoral bone 404, and may be one of many sensors (not illustrated) that are individually activated to send active signals including position information to a computing device either over a communication network or directly via short-range wireless communication (e.g., Bluetooth, Near-Field Communication, etc.). The sensor 402 may be activated by a magnetic switch, and identified or labeled according to a position relative to a plurality of predefined positions, and registered with an interface on the computing device. Together with an interface and a software application, the computing device may calculate the orientation of cutting planes on a tibial plateau of tibia 408, enabling a proper physiological configuration based on individual patient kinematic data. The relation between the ligament balancing tool and sensor 402 together with their positions in a three-dimensional space results in, e.g., a three-dimensional, trajectory system.

After opening the joint and removing at least portions of meniscus 406 and/or cartilage 412, cutting of the tibia 408 is performed to prepare the surface of the for tibia for a tibial baseplate. Prior to selecting an implant, an initial measurement may be performed by using the interface of the software application on the computing device to receive and display values from the dynamic ligament balancing tool and sensor 404. Necessary information for a proper implantation can be presented on the interface by the software application which enables a surgeon to set his cuts based on the initial measurement results.

A user interface may present reference data captured by a ligament balancing tool for bicondylar knee arthroplasty. An initial setting of a chosen tension on the ligament balancing tool may be configured (e.g., 20, 25 or 30 newtons) with the software application. After a calibration process, a three-dimensional avatar comprising a virtual representation of the joint and the dynamic ligament tool may be displayed along with a current flexion. Distance values representative of tension measured by the ligament balancing tool at the medial collateral ligament and the lateral collateral ligament, as well as intercondylar angle valued for femoral rotation of a femoral implant, may be presented at various critical flexion angles.

Relative three-dimensional orientation of the femoral bone and the tibia may also be indicated by the avatar and a current rotation status. Current rotation status may comprise positions indicated by a marker corresponding to the dynamic ligament balancing tool in relation to a marker corresponding to the sensor placed on the femoral bone. By this way, the position of the tibia can be adjusted to a correct position in relation to an initial rotational alignment with respect to the femoral bone sensor by aligning the markers. A current rotation status may show that a marker corresponding to the femur is off alignment with a marker corresponding to the tibia. A distal femur setting recommendation may be generated by the software application based on, for example, angle measurement of preoperative long leg x-rays. A rotational recommendation may also be generated according to a reference measurement.

Using results from the initial measurement, a femoral cutting block can be adjusted to the right position and the femoral cuts may be performed. Additionally, in conjunction with the data presented by the software application, preoperative testing data from a system, such as the previously discussed MacP system and/or using additional ones of sensors, such as sensor 402, in a three-dimensional orientation system, the surgeon is able to improve the accuracy of the cuts by sensor guided planes and surveillance of the depth of the cut to avoid soft tissue damage. For example, the depth of the cut may be guided by a sensor mounted to a saw to show the borders in case of limited visibility (for example, in minimal invasive surgery procedures). After cutting the femur, a femoral trial implant may be placed on the femoral surface and a control measurement may be performed using the interface and software application executing on the computing device.

The user interface may also present control data captured by the ligament balancing tool for bicondylar knee arthroplasty. A three-dimensional avatar comprising a virtual representation of the joint, the trial implant, and the dynamic ligament tool may be displayed along with a current flexion. The avatar may further indicate balancing condition based on a rendered color of the dynamic ligament balancing tool. For example, green may indicate acceptable balancing, yellow may indicate borderline balancing, and red may indicate incorrect balancing. Distance values representative of tension measured by the ligament balancing tool at the medial collateral ligament and the lateral collateral ligament may be presented at various critical flexion angles. A current rotation status comprises positions indicated by a marker corresponding to the dynamic ligament balancing tool in relation to a marker corresponding to the sensor placed on the femoral bone, which in the illustrated example, shows that the two markers are in a correct position or alignment. Based on the values provided by the control data screen, definite implants can be fixed. In case of any mismatch, imbalance or asymmetry, correctional cuts or slight soft tissue releases can be performed to reach target parameters.

The avatar may also indicate that a selected trial implant produces a balancing condition that is incorrect and renders the dynamic ligament balancing tool in red color. Additionally, a current rotation status comprises positions indicated by a marker corresponding to the dynamic ligament balancing tool in relation to a marker corresponding to the sensor placed on the femoral bone may show that the two markers are incorrectly positioned and out of alignment. To correct the imbalance, a recommended inlay thickness may be determined and presented based on geometrical data of available implants.

Pre-, intra- and postoperative measurement data (e.g., reference data, control data, etc.) may be stored by the computing device to a cloud database that is secured by an access hierarchy in accordance with data protection regulations. The disclosed system may further identify an individual kinematic situation of a specific patient including the knee motion and range of motion as well as individual limitations and abilities that can affect the ideal configuration of components of the knee replacement. The pre-, intra- and postoperative measurements can be used for quality control and can be connected to the previously discussed MacP system.

The patellofemoral joint is a rarely appreciated part of the knee joint. Although one third of knee problems result from patellofemoral arthrosis, often combined with valgus deformities and valgus arthrosis, no dedicated system for determining the right procedure for the patellofemoral joint is available. According to one embodiment, a dynamic patellofemoral balancing system is disclosed. The patellofemoral balancing system can enable a surgeon to evaluate positioning of a cutting plane (e.g., for replacing the patella surface) and pressure in this part of the knee joint during total knee arthroplasty. As such, tilting and mispositioning can be avoided as well as anterior knee pain can be reduced.

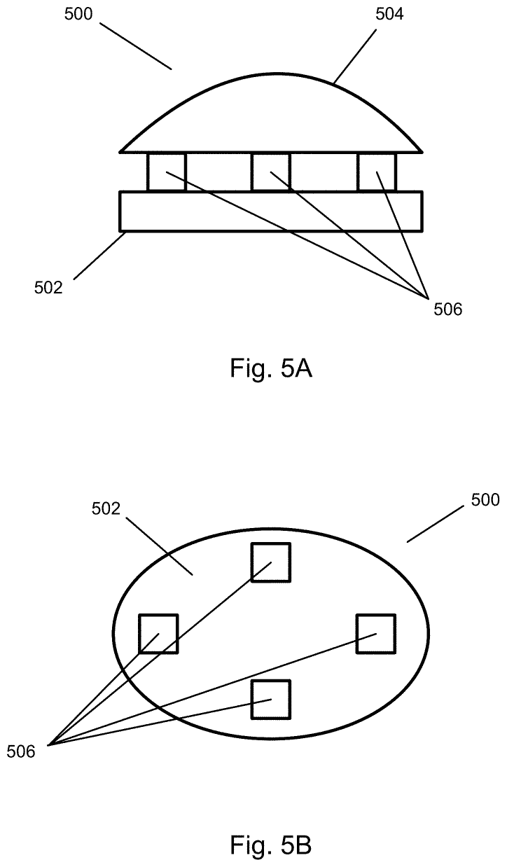

FIG. 5A presents a side view of a patella balancing device for patella replacement according to an embodiment of the present invention. The patella balancing device 500 is configured to identify thickness, position, and pressure of a patellofemoral joint over an entire range of motion of the knee. The patella balancing device 500 includes baseplate 502, hemisphere 504 and spring coils 506. The baseplate 502 may include a surface that rests against the surface of the patella that faces the femur. The hemisphere 504 may include a rounded exterior surface for interfacing with the femur.

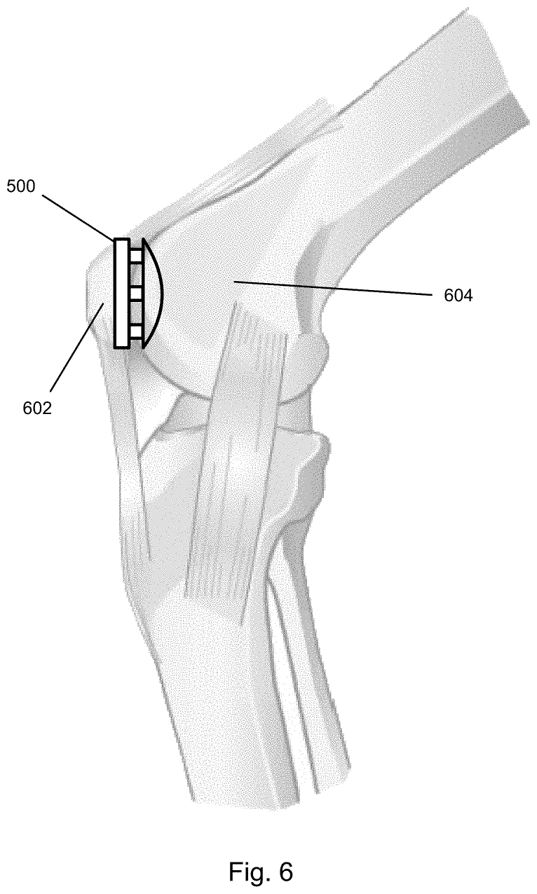

FIG. 5B presents an exposed top view of the patella balancing device without the hemisphere 504. Spring coils 506 are embedded on or affixed to baseplate 502. The hemisphere 504 is configured above the spring coils 506 such that baseplate 502 may remain stationary while hemisphere 504 is adjustable in relation to the baseplate 502 by spring coils 506 which can be controlled by a client device. FIG. 6 presents an exemplary configuration of the patella balancing device according to an embodiment of the present invention. After a calibration process with the client device, the patella balancing device 500 may be situated within a patellofemoral joint in between patella 602 and femur 604, as illustrated in FIG. 6.

The patella balancing device 500 may be activated by the client device to control the spring coils 506 to lift or lower hemisphere 504. The spring coils 506 include peg structures that operate with the spring coils 506. The patella balancing device 500 may tension up the soft tissue envelope surrounding the bone to a defined tension by lifting and lowering the hemisphere 504 with the spring coils 506. Each of the spring coils 506 may be moved or operated either independently or collectively. The patella balancing device 500 further includes a wireless electronic module (e.g., Bluetooth) for connection to a client device and a radio-frequency identification (“RFID”) chip for position data acquisition by the client device. The client device may obtain and tune the distance between the baseplate 502 and hemisphere 504 by adjusting the spring coils 506. After reaching a correct or desired position, the spring coils 506 may be locked for checking and released for testing.

The distance between the baseplate 502 and hemisphere 504 may be set based on an appropriate tension value (e.g., 6 mm at 20 newtons) that may be measured by patella balancing device 500 and transmitted to the client device. Particularly, distance values at each of the spring coils 506 may be used as a reference for implantation of a femoral prosthesis. The spring coils 506 further include sensors that may record and transmit measurement data to client device. In particular, the sensors may comprise hall sensors, gyro sensors, and RFID sensors that measure distance, tension, and three-dimensional orientation of the patella balancing device 500 in relation to specific anatomical landmarks marked by positioning sensors, as discussed above. The measurement data may be used by the client device to generate a display of measurements in quadrants of the patella balancing device 500 corresponding to the spring coils 506. The measurements data may also be used by the client device in combination with data from the sensors placed at the specific anatomical landmarks to generate a three-dimensional joint movement model that shows prosthesis positioning and any deviation which may be outside a tolerable range.

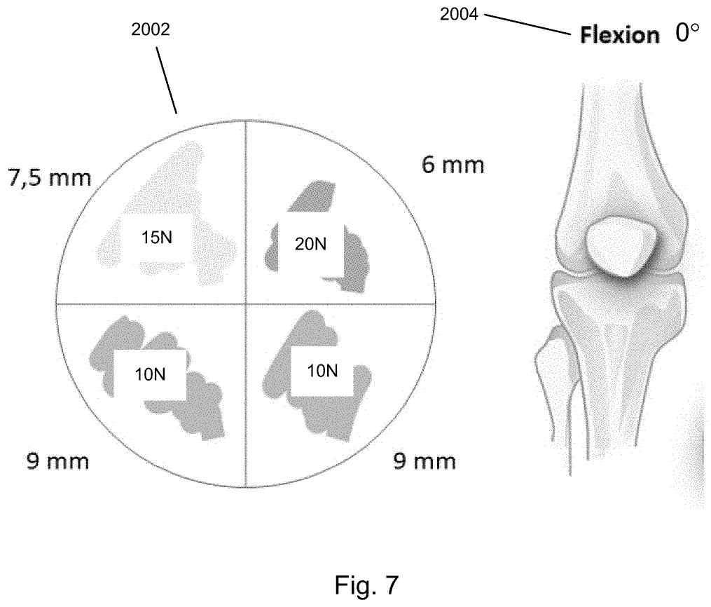

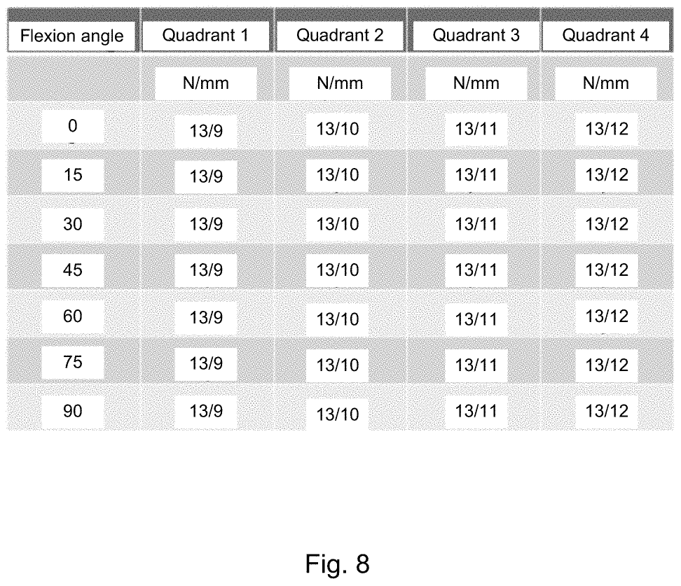

FIGS. 7 and 8 present exemplary interfaces for the patella balancing device according to an embodiment of the present invention. The patella may be prepared and mounted with the patella balancing device to perform a measurement procedure over an entire range of motion of the knee. Measurement quadrants 2002 display force/load values within a circle representative of the patella balancing device 500 and distance values are displayed outside the circle. Flexion angle 2004 is also displayed to indicate a joint angle corresponding to the values of measurement quadrants 2002. Each of the quadrants may also include color indications of proper intra-operative load distribution (e.g., green indicates acceptable, yellow indicates borderline balancing, and red indicates incorrect balancing) when a knee inserted with the patella balancing device 500 is moved over the entire range of motion of the knee. Additionally, a table (FIG. 8) may also be presented including the force/load values and the distance values over predetermined flexion angles.

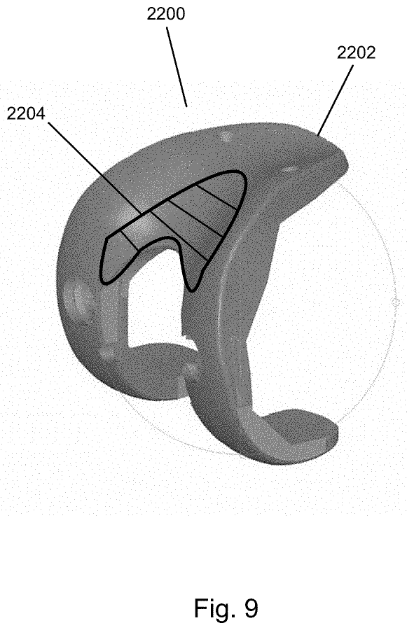

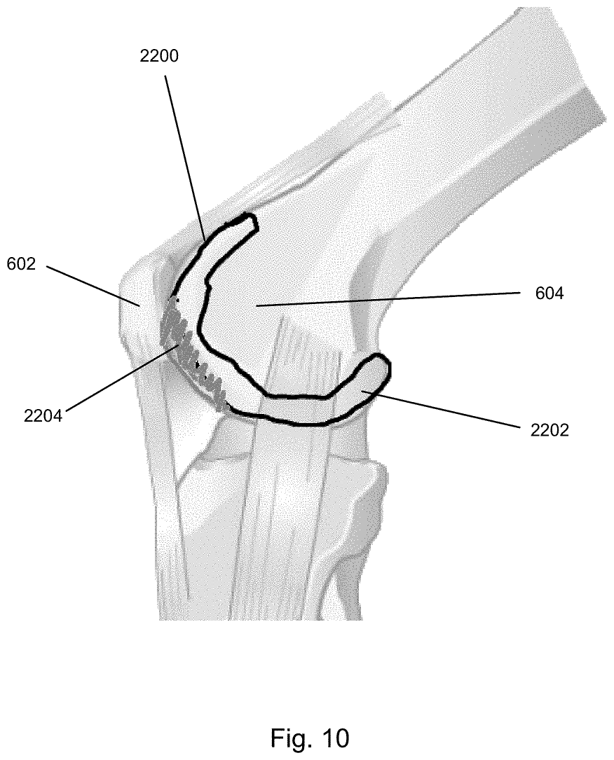

FIG. 9 presents a femoral balancing device for femur replacement according to an embodiment of the present invention. The femoral balancing device 2200 comprises a femoral implant 2202 and an embedded sensor 2204. The embedded sensor 2204 may be inserted and removed from a notch of the femoral implant 2202. The notch comprises a concave portion of the femoral implant 2202 that makes direct contact with the patella. The embedded sensor 2204 may be a removable component from femoral implant 2202 and is compatible with a plurality of femoral implant sizes. In the course of performing a total knee arthroplasty procedure and performing a femoral “4-in-1” cut, femoral balancing device 2200 may be placed on a femur 604, as illustrated in FIG. 10. Embedded sensor 2204 may measure and detect position, alignment and pressure against the surface of a patella 602. By measuring the pressure, the right congruence between the femoral implant 2202 and the patella 602 can be achieved. The embedded sensor 2204 may transmit data corresponding to the measurement and detection to a computing device including an interface for displaying the measurement data.

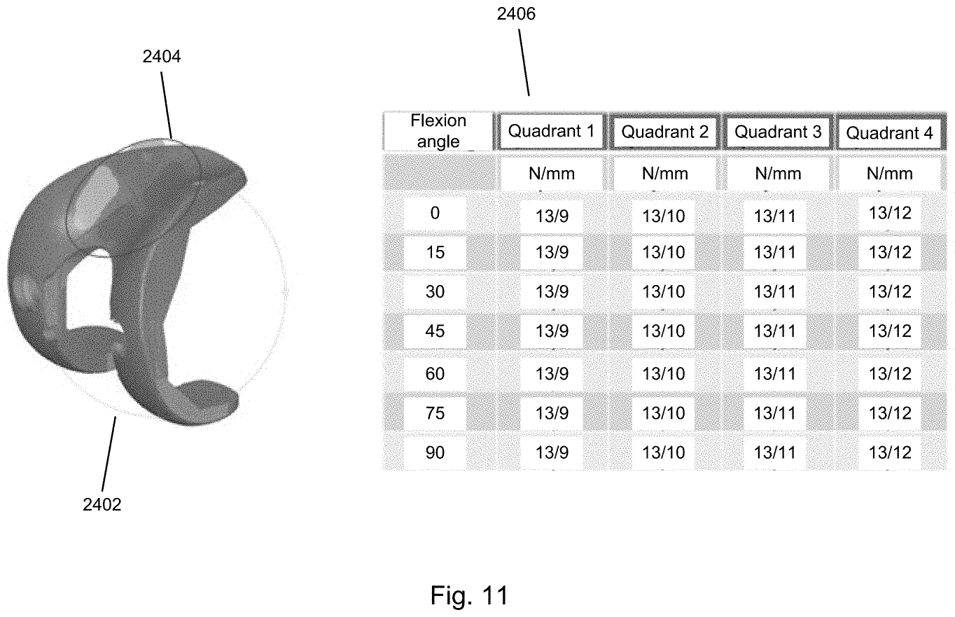

FIG. 11 illustrates an exemplary interface for femur replacement according to an embodiment of the present invention. The interface may include an avatar 2402 comprising a representation of the femoral balancing device 2200. The avatar 2402 may include measurement quadrants 2404 which indicates pressure in a loading zone of the femoral implant 2202 that is measured by embedded sensor 2204. The pressure may be indicated by color (e.g., green as acceptable, yellow as borderline, and red as too high). Additionally, pressure detected at the measurement quadrants 2404 may be recorded for the entire range of motion of the knee. The interface may further include a table 2406 including force distribution on the femoropatellar surface over various flexion angles at quadrants corresponding to the embedded sensor 2204.

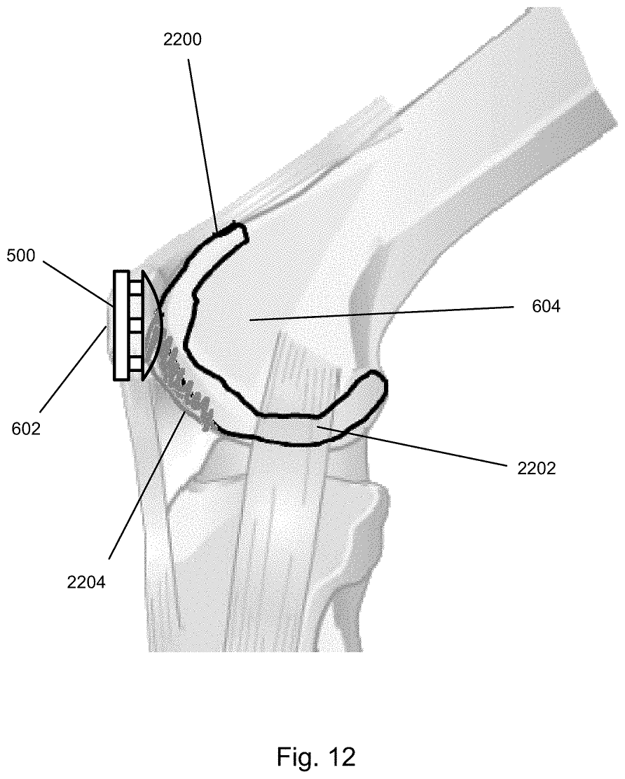

FIG. 12 illustrates patellofemoral balancing devices for patella and femur replacement according to an embodiment of the present invention. For a procedure that includes patella resurfacing (or replacement) in addition to femur replacement, femoral balancing device 2200 may be placed on a femur 604 in combination with the patella balancing device 500 situated in between patella 602 and femoral balancing device 2200. Parallel measurements of pressure, distance, tension, and load distribution may then be performed at the femur 604 and patella 602 with the patellofemoral balancing devices.

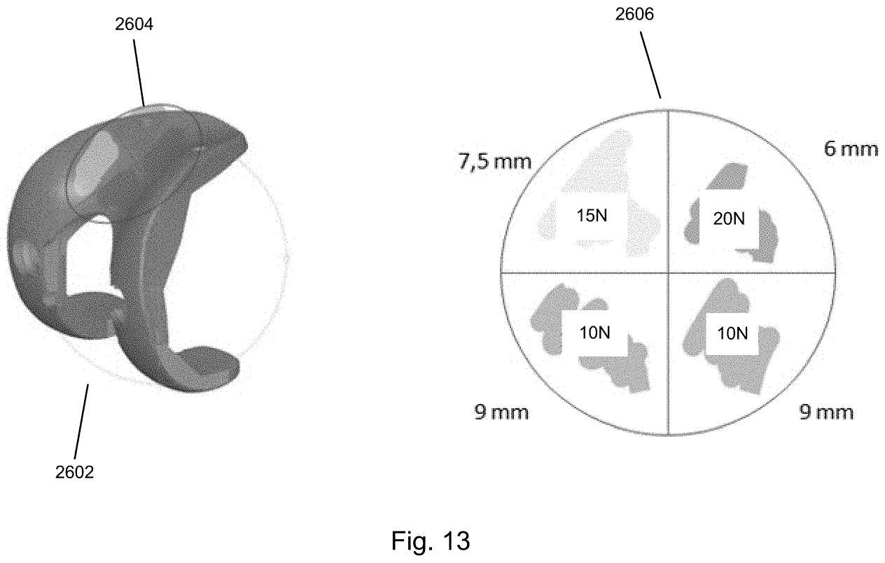

FIG. 13 illustrates an exemplary interface for patella and femur replacement according to an embodiment of the present invention. The interface may include an avatar 2602 comprising a representation of the femoral balancing device 2200. The avatar 2602 may include measurement quadrants 2604 which indicate pressure in the loading zone of the femoral implant 2202 measured by embedded sensor 2204. The interface may also include measurement quadrants 2606 which display force/load values within a circle representative of the patella balancing device 500 and distance values are displayed outside the circle. Each of the quadrants 2604 and 2606 may also include color indications of proper intra-operative load distribution (e.g., green indicates acceptable, yellow indicates borderline balancing, and red indicates incorrect balancing). The pressure and loads detected at the measurement quadrants 2604 and 2606 may be recorded for the entire range of motion of the knee and displayed.

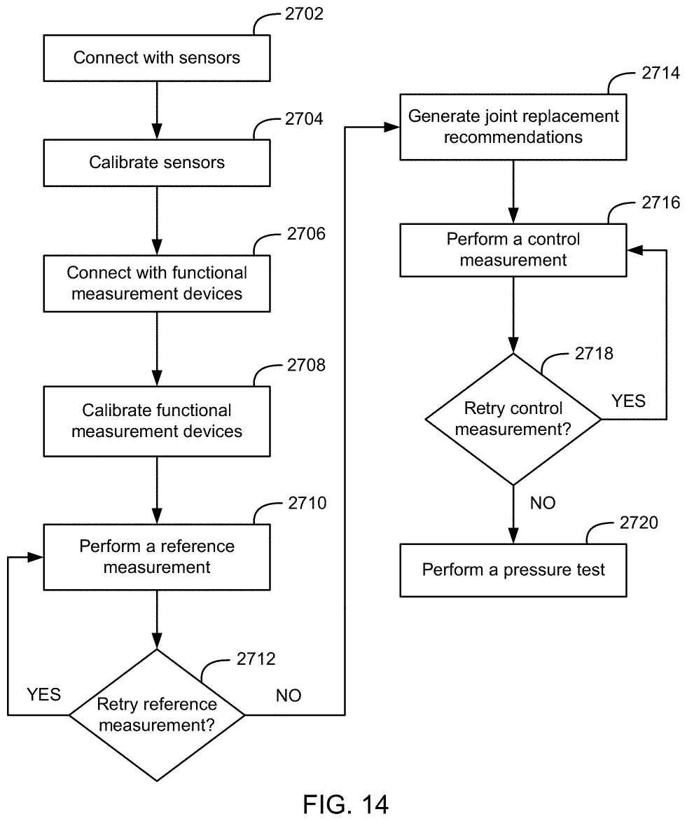

FIG. 14 presents a flowchart of a method for guiding knee replacement procedures according to an embodiment of the present invention. Sensors may be placed at a joint of an individual being treated, such as on an anterior surface of a femur bone at a knee joint. A client device executing an application for guiding a knee replacement procedure is connected with one or more sensors, step 2702. Connecting the client device with the one or more sensors may include a pairing procedure. For example, a button on a given sensor may be pressed to cause the sensor to broadcast a pairing signal. The client device may detect the pairing signal from the given sensor and establish a connection with the given sensor. A pairing number or identifier and operating status (e.g., battery level and signal strength) may be retrieved from the given sensor by the client device for display on an interface.

The sensors are calibrated, step 2704. The sensors may be calibrated with the client device by performing several operations such as, moving forward and backward, flexing and extending a joint attached to the one or more sensors, or rotating the joint, as instructed by the client device. The client device may display an indicator to indicate that a calibration of the sensors was successful.

The client device is connected with one or more functional measurement devices, step 2706. A functional measurement device may be paired with the client device by switching on the functional measurement device. The functional measurement device may be switched on by physically depressing and holding paddles on the functional measurement device. Upon switching on, the functional measurement device may broadcast a pairing signal. The client device may detect the pairing signal from the functional measurement device and establish a connection with the functional measurement device. A pairing number or identifier and operating status (e.g., battery level and signal strength) may be retrieved from the functional measurement device by the client device for display on an interface.

The functional measurement devices are calibrated, step 2708. The functional measurement devices may be calibrated with the client device by releasing the paddles after depressing them to pair the functional measurement devices with the client device. The client device may display an indicator to indicate that calibration of the functional measurement devices is successful. The functional measurement devices may be inserted within the knee joint and mounted above a tibial surface.

A reference measurement is performed, step 2710. The reference measurement may include a preoperative testing procedure that is directed by the client device. The preoperative testing procedure may comprise a series of testing that measures the operation and characteristics of the joint, e.g., prior to a femoral cut, based on measurements taken by the sensors and functional measurement devices, which collectively represent functional stability of the joint. The reference measurement may comprise a recording of data values received from the sensors and the functional measurement devices during which the user may be instructed to, for example, fully flex and extend a joint (e.g., from 0° to 90°). The data values may include paddle heights (or displacement) at the medial and lateral condyles along with corresponding pressures or tension at given angles, such as 0°, 30°, 60°, and 90°.

The client device may provide an option for the user to retry the reference measurement, step 2712. The reference measurement may be repeated to verify reference measurement data or to re-measure after surgical or sensor/functional measurement device adjustments. The client device may return to step 2710 to retry the reference measurement.

If the reference measurements are satisfactory, reference measurement is not retried and the client device proceeds to generate joint replacement recommendations based on the reference measurement, step 2714. Generating the joint replacement recommendations may include determining and/or calculating one or more of ideal joint planes, alignment, cutting planes, cutting thickness, distal femur angle, femoral rotation, implant types and sizes that are individualized to the patient based on the comparison to regain functionality and range of motion.

The client device may further modify or improve the joint replacement recommendations based on data of healthy people with normal joint function as well as surgical data of other patients. For example, an analysis engine may determine optimal knee replacement parameters for a particular patient according to their preoperative testing and evaluations in addition to their sex, age, weight, height, and other physical factors to be compared with other patients. The client device may render on an interface a recommendation including a comparison of a healthy side and an affected side, and range of motion.

In the case of surgery, measurements similar to the preoperative testing procedure associated with step 2710 may be repeated during surgery to compare pre-, intra-, and postoperative status. Such measurements may include assessing joint stability and replicating the stability in joint replacement surgery. The disclosed system may comprise a computing system that acquires muscular data to perform gait analysis and load distribution analysis for configuring the optimal post-operative treatment protocols in accordance with individual capabilities and muscle function, which is discussed in further detail in commonly-owned U.S. Patent Application No. 63/176,079, entitled “SYSTEM AND METHOD FOR FUNCTIONAL STABILITY PLANNING OF REPLACEMENT JOINTS,” filed on Apr. 16, 2021, which is incorporated herein by reference.

A control measurement is performed, step 2716. The control measurement may be performed, for example, after performing femoral cuts based on the joint replacement recommendations. The control measurement may comprise additional testing that measures operation and characteristics of the joint after performing a procedure based on the joint replacement recommendations. The control measurement may comprise a recording of data values received from the sensors and the functional measurement devices during which the user may be instructed to, for example, fully flex and extend a joint (e.g., from 0° to 90°). Data from the control measurement may be compared with the reference measurement on the client device.

The client device may provide an option for the user to retry the control measurement, step 2718. The control measurement may be repeated to verify control measurement data or to re-measure after surgical or sensor/functional measurement device adjustments. The client device may return to step 2716 to retry the control measurement.

If the control measurements are satisfactory, control measurement is not retried and the client device proceeds to perform a pressure test, step 2720. The pressure test may comprise a test to measure anterior and posterior loading, balance, and kinematic behavior at the medial and lateral condyles through a range of motion. To perform the pressure test, paddles of the functional measurement devices may be locked at a functional/reference height and inserted into the knee joint. Data values may be recorded while moving the knee joint, e.g., from 0° to 90° of flexion and repeating as necessary during the pressure test. In particular, force distribution of the medial and lateral condyles that is measured on the paddles of the functional measurement devices.

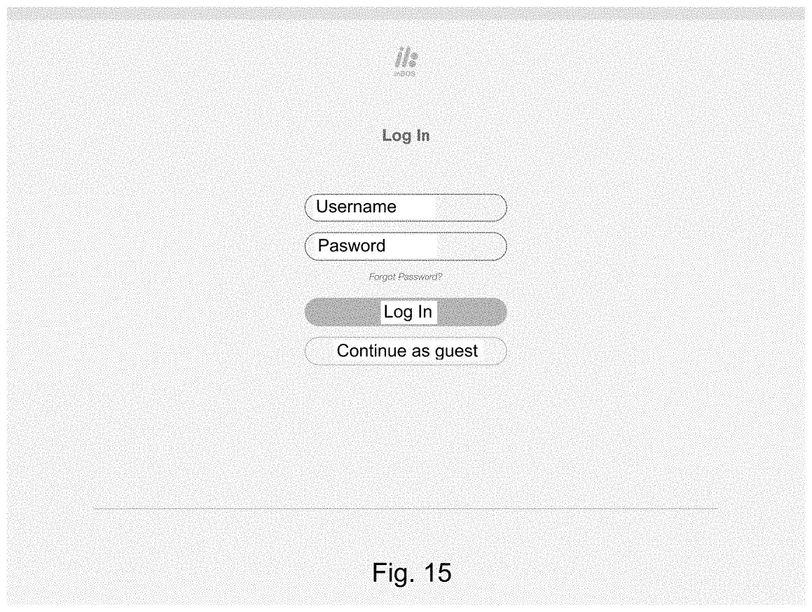

FIGS. 15 through 45 present exemplary user interfaces of a functional stability measurement and analysis system according to an embodiment of the present invention. A user may access a functional stability measurement and analysis system by logging in from a client device through a log in screen as illustrated in FIG. 15. The user may provide a username and password to log in. Alternatively, the user may log in as a guest.

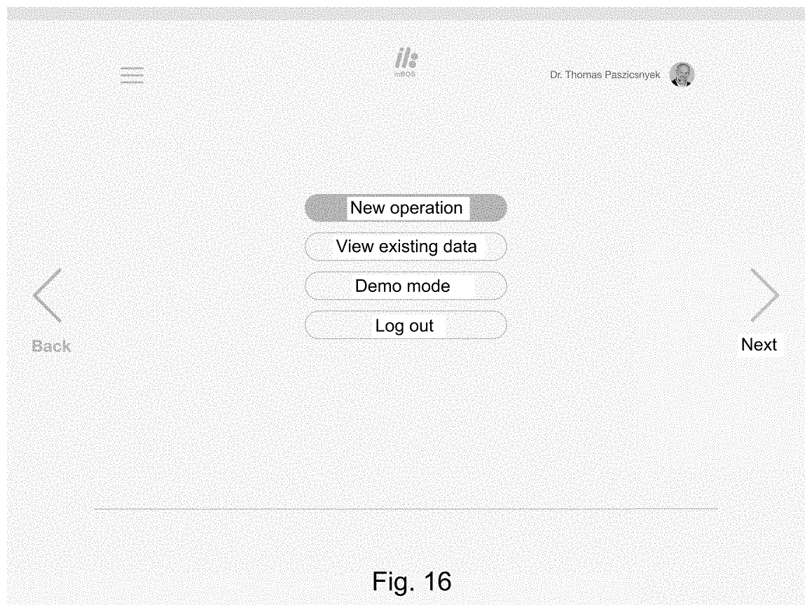

FIG. 16 presents a menu of options for a functional stability measurement and analysis system according to an embodiment of the present invention. The menu includes an option to create a new operation file, view existing patient data, view a demo mode, and log out. Creating a new operation file may allow a user to enter patient information and utilize various surgical tools and functionalities.

The user may also view existing patient data comprising operation files including preoperative and postoperative data that may be stored and retrieved from storage device 110. The preoperative measurements and evaluations data may include scans, testing, physiological analysis, and surveys to provide anatomical situations, kinematic situations, requests, and demands. The postoperative measurements and evaluations data may be similar in type to the preoperative measurements and evaluations data but is instead recorded after certain periods following surgery.

The demo mode may be accessed by the user to receive instructions for using the functional stability measurement and analysis system.

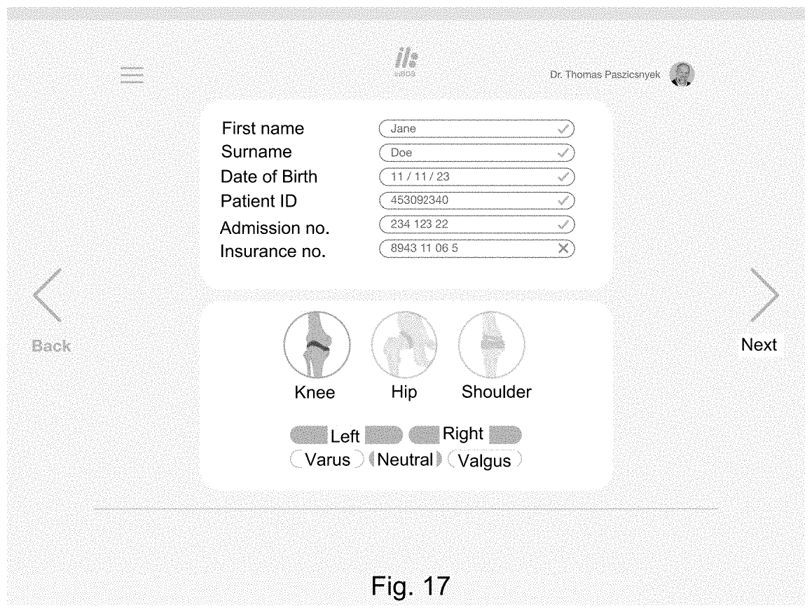

FIG. 17 presents an exemplary operation file according to an embodiment of the present invention. An operation file comprises patient information and surgical procedure information. Patient information includes patient name, date of birth, patient ID, admission number, and insurance number. Surgical procedure information includes an operation location (selectable from knee, hip, or shoulder), an anatomical side (left or right). For a knee procedure, an alignment may also be selected (varus, neutral, or valgus).

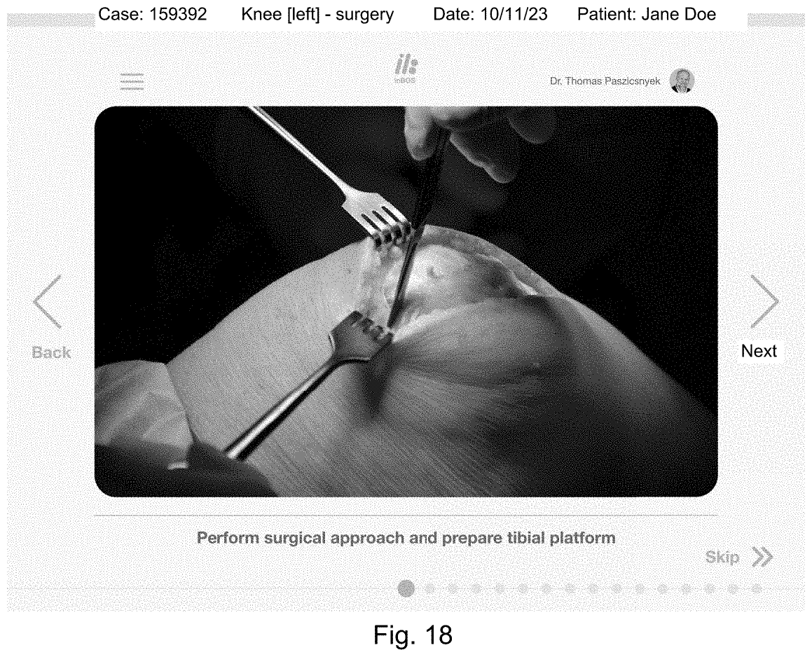

According to one embodiment, the disclosed functional stability measurement and analysis system may be used for guiding a surgical procedure. For example, a knee to be operated on is prepared for measurements by cutting and preparing a tibial surface (FIG. 18).

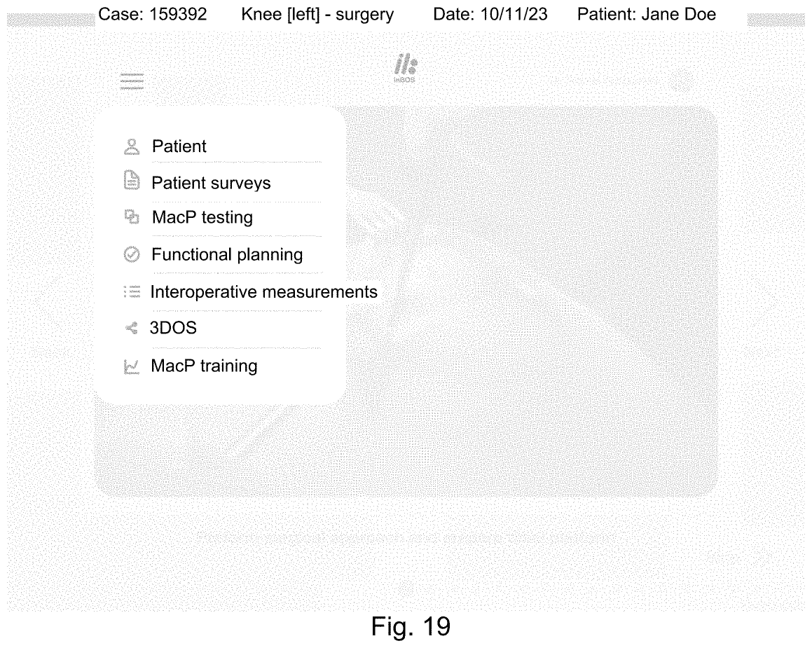

FIG. 19 presents exemplary surgical tools and functionalities of a functional stability measurement and analysis system according to an embodiment of the present invention. A plurality of features may be provided for a given operation file. The user may access patient data, the patient's surveys, muscle activity controlled physiotherapy (“MacP”) testing, functional planning, interoperative measurements, a three-dimensional orientation system (“3DOS”), and MacP training. Patient surveys may comprise surveys for preferences and lifestyles (e.g., current activities and capabilities) and demands (e.g., desired kinds of activities and capabilities). MacP testing may be used to identify the patient's kinematic situation and range of motion as well as individual limitations and abilities by measuring and examining muscular ability using a MacP system.

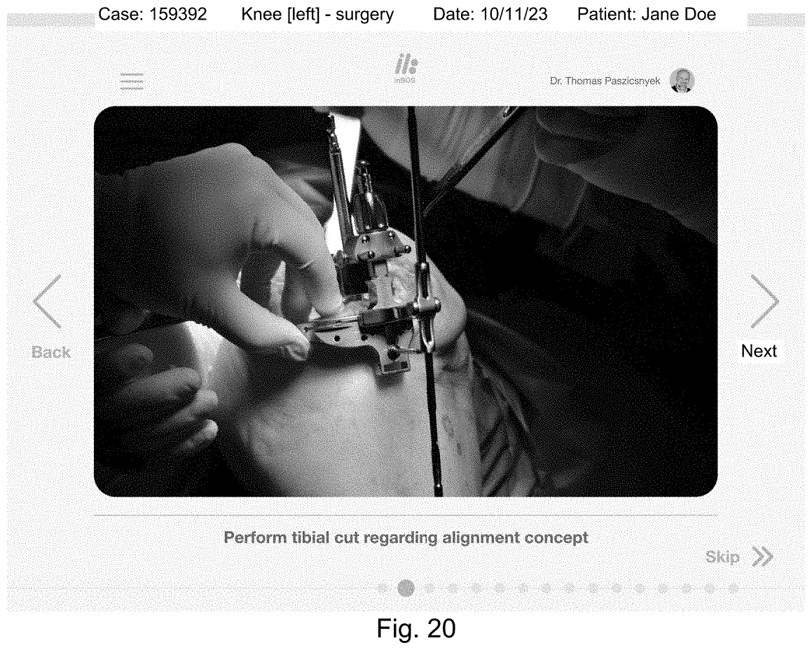

Functional planning may comprise retrieving data sets such as, sensor data X-Rays, gait analyses, load distribution analysis, and motion analysis. Based on the data sets, functional planning data may be generated including preoperative and postoperative joint stability and functionality, and recommended implant sizes to achieve joint replacement that is satisfactory in performance. 3DOS may comprise using sensors to aid and improve the accuracy of cuts by providing sensor guided planes and surveillance of the depth of a cut to avoid soft tissue damage. For example, 3DOS may be used to perform a tibial cut according to a provided alignment (FIG. 20). MacP training may comprise retrieving status of muscular and coordinate ability and creating a rehabilitation training plan based on measurements of muscle activity of an affected joint.

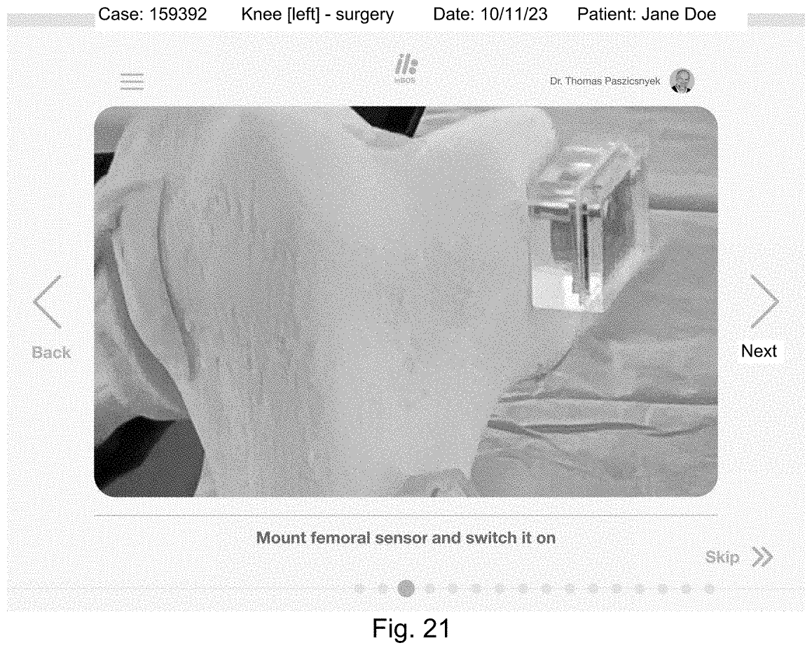

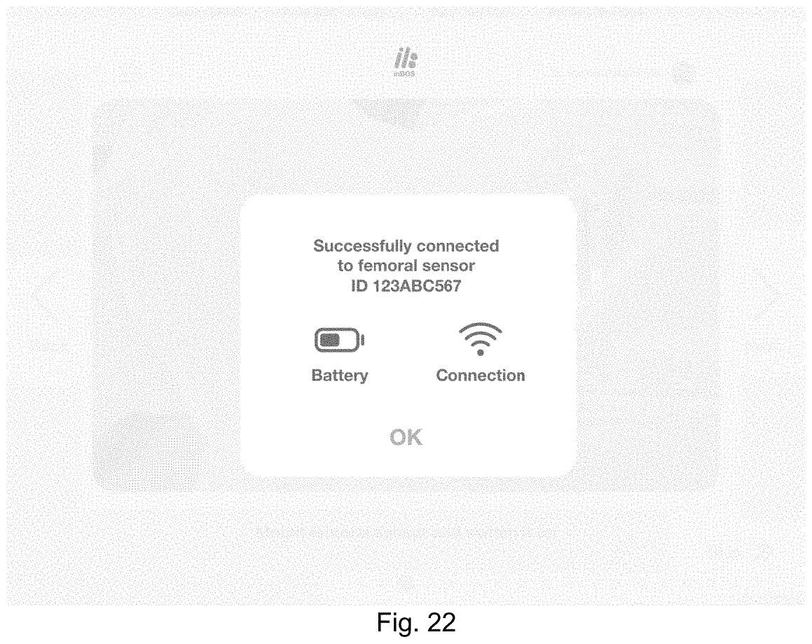

Interoperative measurements may comprise measuring joint tensioning and load while performing a surgical procedure. To perform the interoperative measurements, in the case of knee surgery, a sensor may be mounted on the femur bone of the knee being operated on, e.g., on the ventral surface of the femur (FIG. 21). The sensor may include a magnetic switch that activates the sensor on upon contact with a magnetic surface (e.g., fixed on a bone surface). The client device may be connected to the sensor by receiving a pairing signal from the sensor. A plurality of additional sensors may also be mounted at several locations surround the operated joint for use with 3DOS. FIG. 22 presents a confirmation message of a successful connection to the femoral sensor. A pairing number or identifier and operating status (e.g., battery level and signal strength) may be retrieved from the given sensor by the client device and displayed in the confirmation message.

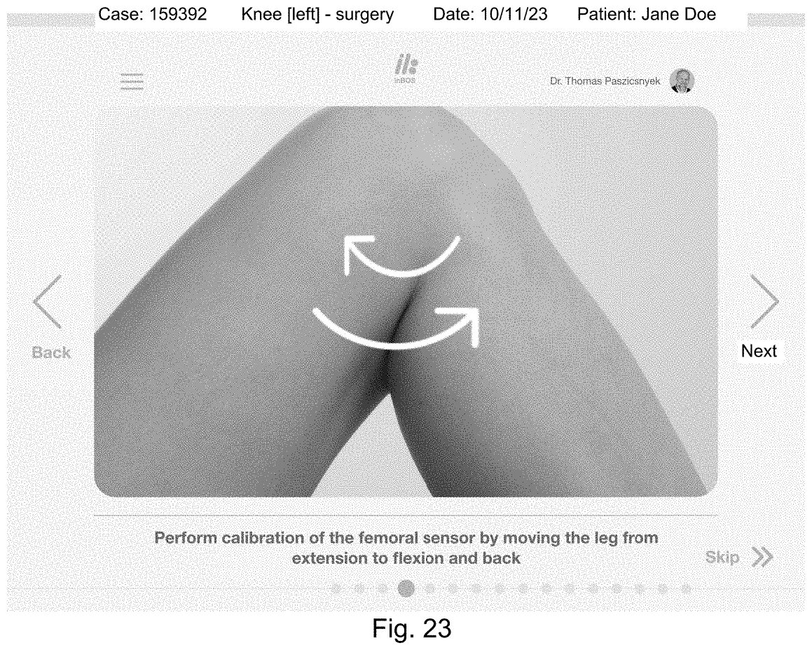

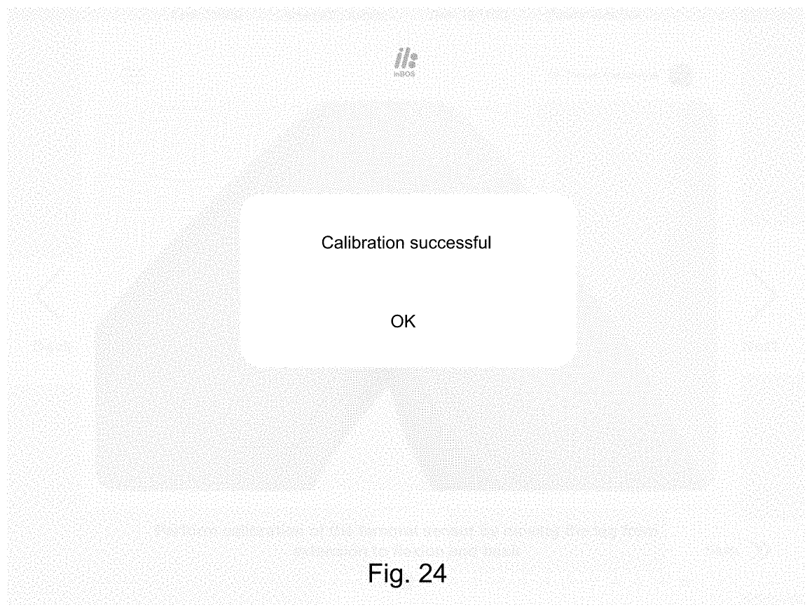

The sensor may be calibrated with the client device by performing several operations such as, moving forward and backward, flexing and extending a joint attached to the sensor, or rotating the joint, as instructed by the client device. As shown by FIG. 23, a client device interface provides instructions for calibrating the femoral sensor by moving the leg from extension to flexion and back. The client device may display a confirmation message (FIG. 24) to indicate that a calibration of the sensor was successful.

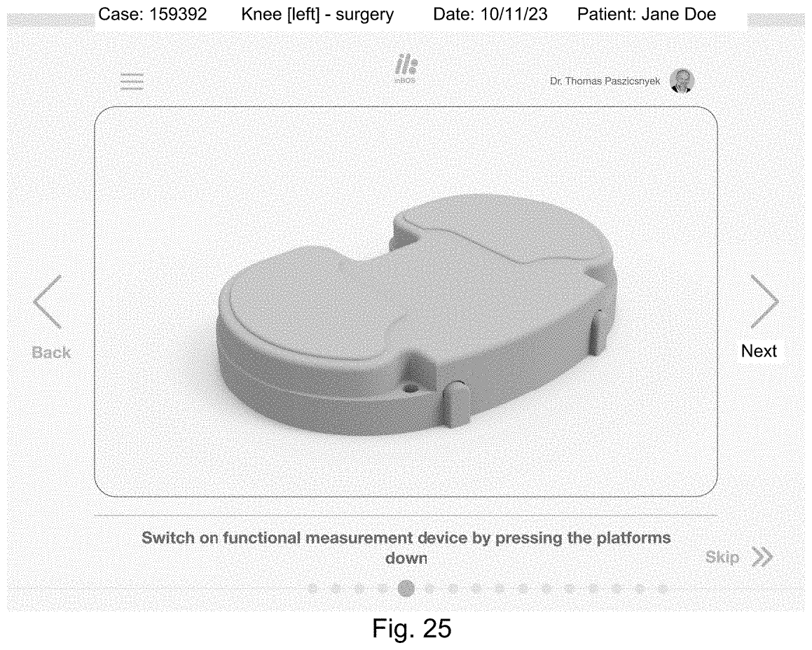

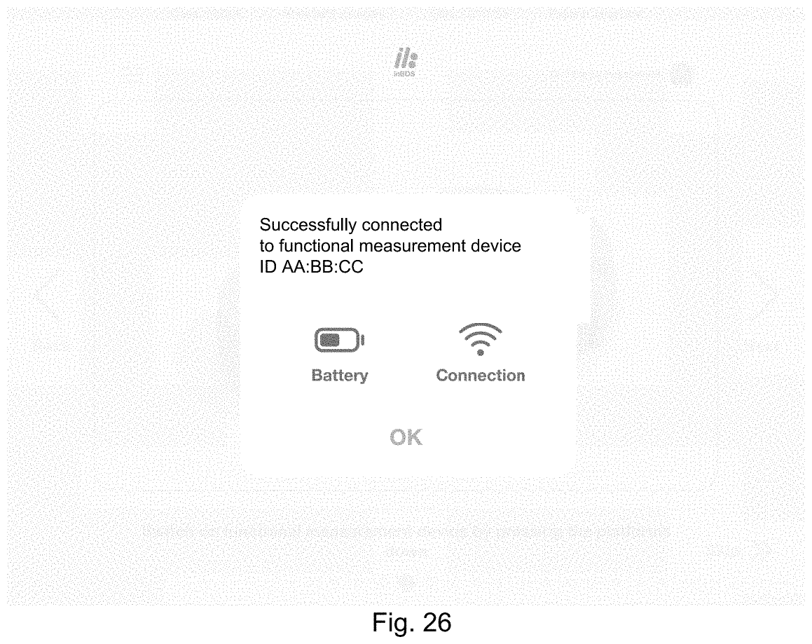

The client device may also be connected with one or more functional measurement devices. A functional measurement device may be connected to the client device by switching on the functional measurement device. FIG. 25 presents a client device interface providing instructions for switching on a functional measurement device according to an embodiment of the present invention. The functional measurement device can be switched on by pressing and holding the paddles down. The client device may detect a pairing signal from the functional measurement device and establish a connection with the functional measurement device. FIG. 26 presents a confirmation message of a successful connection to the functional measurement device. A pairing number or identifier and operating status (e.g., battery level and signal strength) may be retrieved from the functional measurement device by the client device for display on an interface.

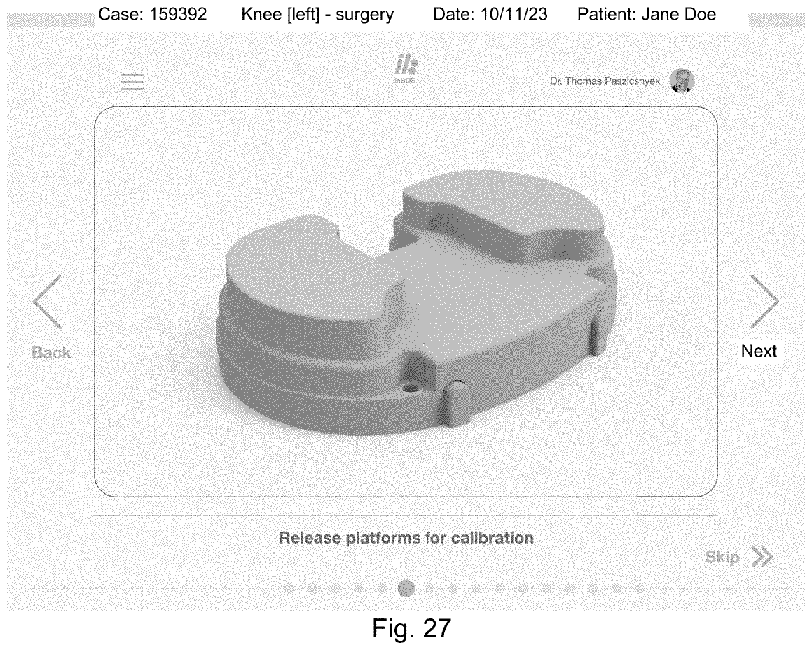

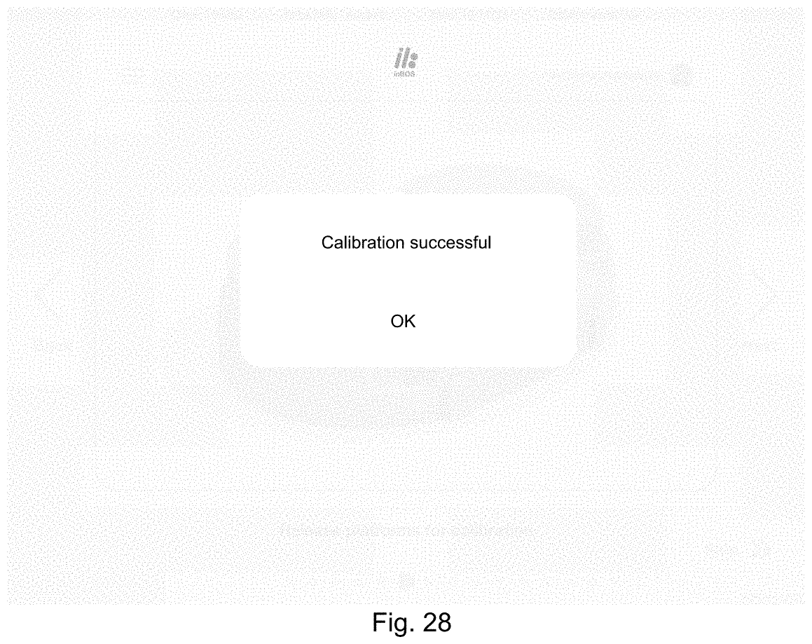

FIG. 27 presents a client device interface providing instructions for calibrating a functional measurement device. The functional measurement device may be calibrated with the client device by releasing the paddles of the functional measurement device. The client device may display an indicator to indicate that calibration of the functional measurement devices is successful (FIG. 28).

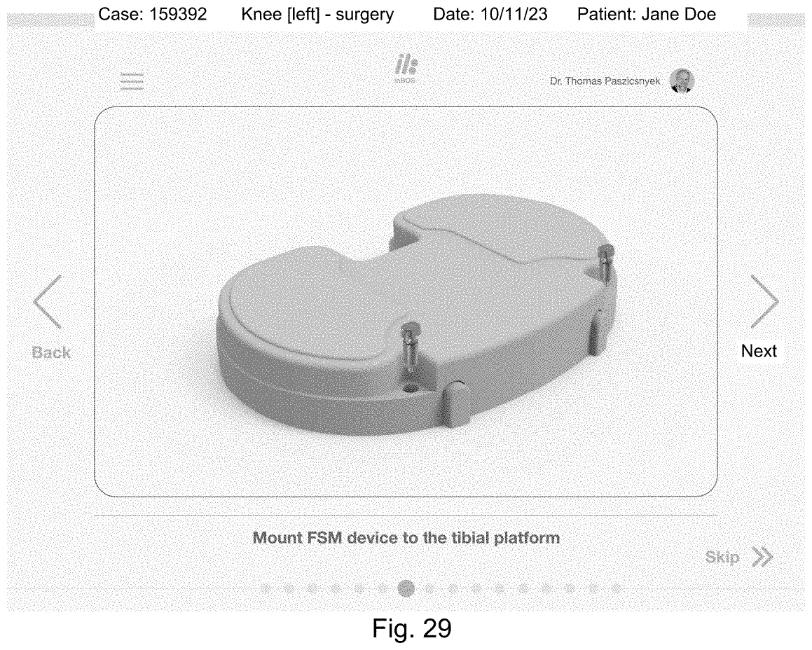

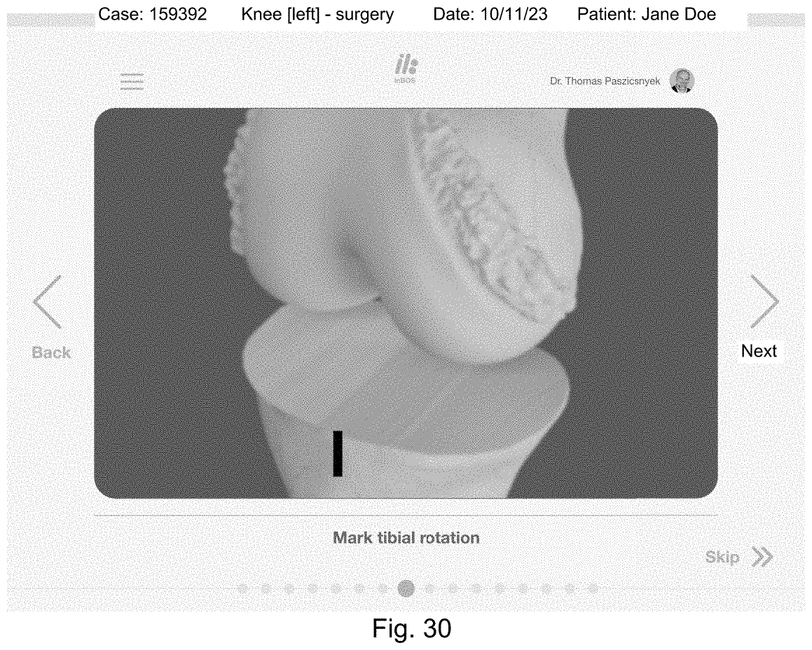

FIG. 29 presents a client device interface providing instructions for mounting a functional measurement device according to an embodiment of the present invention. The functional measurement device may be mounted via pins or anchoring devices on a tibial surface. FIG. 30 presents a client device interface providing instructions for marking of tibial rotation according to an embodiment of the present invention. The tibial bone surface at the joint may be marked at the medial border of the tuberosity to provide a mounting reference for the functional measurement device as well as prosthesis.

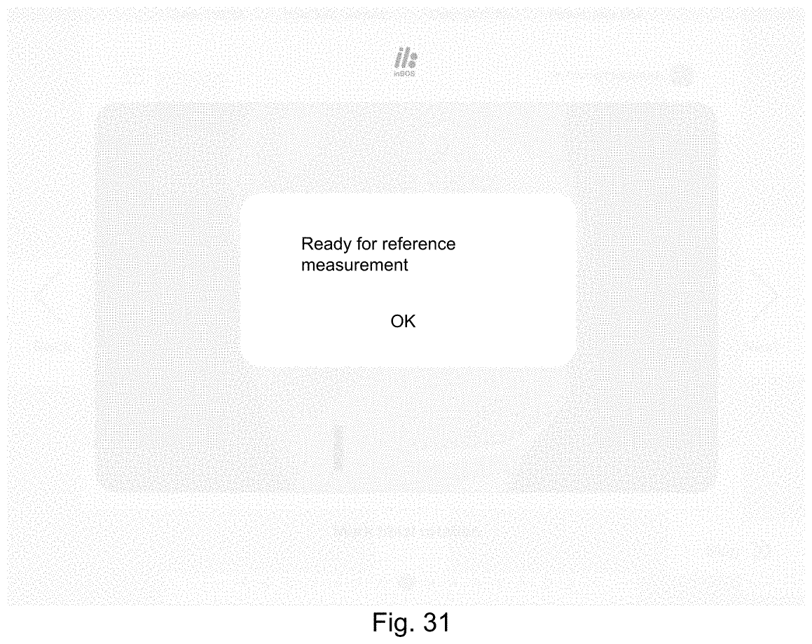

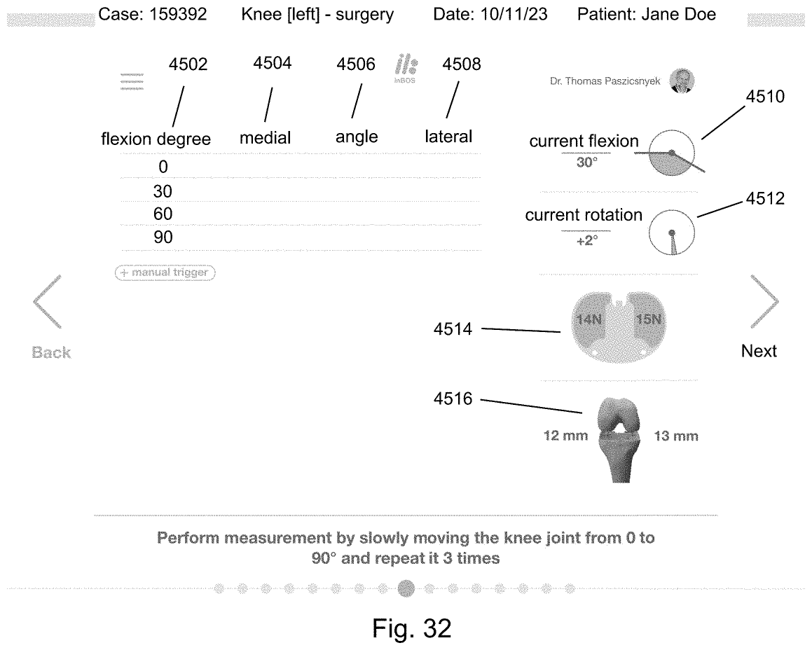

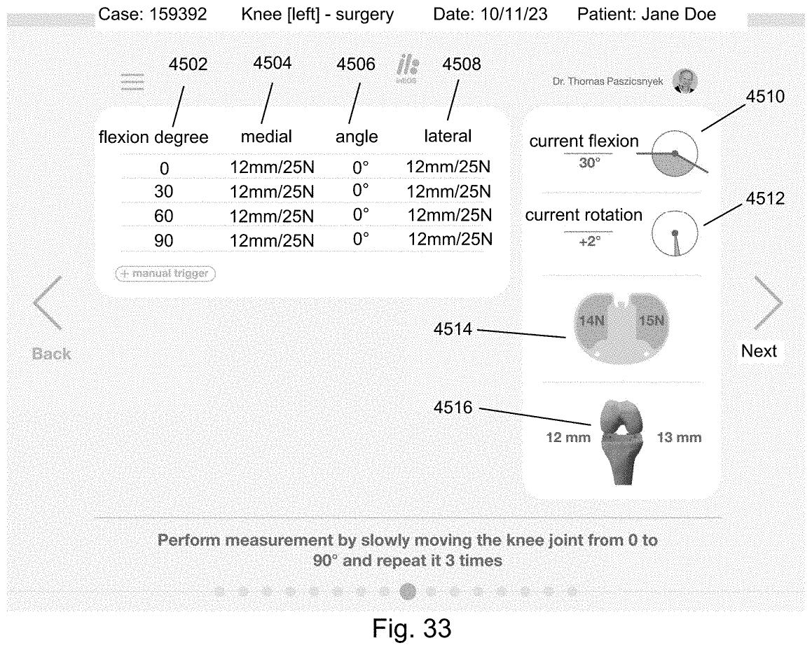

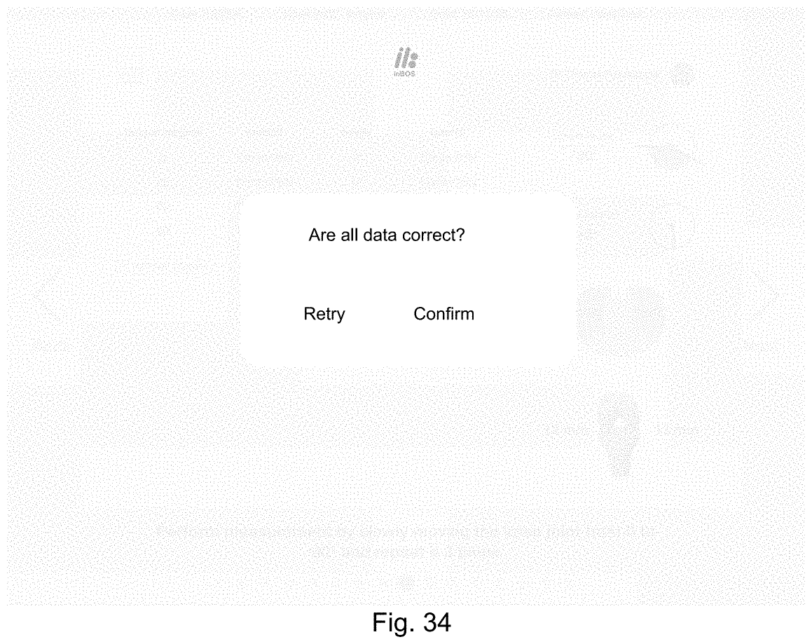

The client device may indicate that the system is ready for performing reference measurements (FIG. 31). FIGS. 32 and 33 present exemplary interfaces for performing reference measurements according to an embodiment of the present invention. In the illustrated embodiment, the client device instructs the user to move the knee joint from 0° to 90° and repeat the procedure an additional three times. Reference measurement data from the functional measurement device may be recorded and rendered on the client device showing flexion degree 4502, paddle distance/force at the medial collateral ligament 4504 and the lateral collateral ligament 4508, intercondylar angle 4506, current flexion 4510, current rotation 4512, functional measurement device paddle loads 4514, and functional measurement device paddle distances 4516. The client device may provide the user an option to proceed by confirming that the measurement data is correct or retry performing reference measurements (FIG. 34).

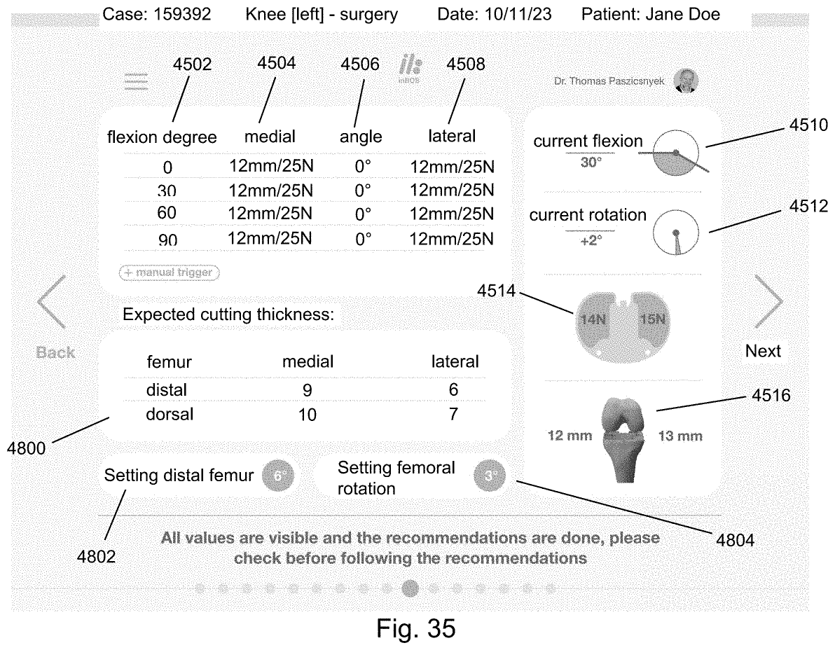

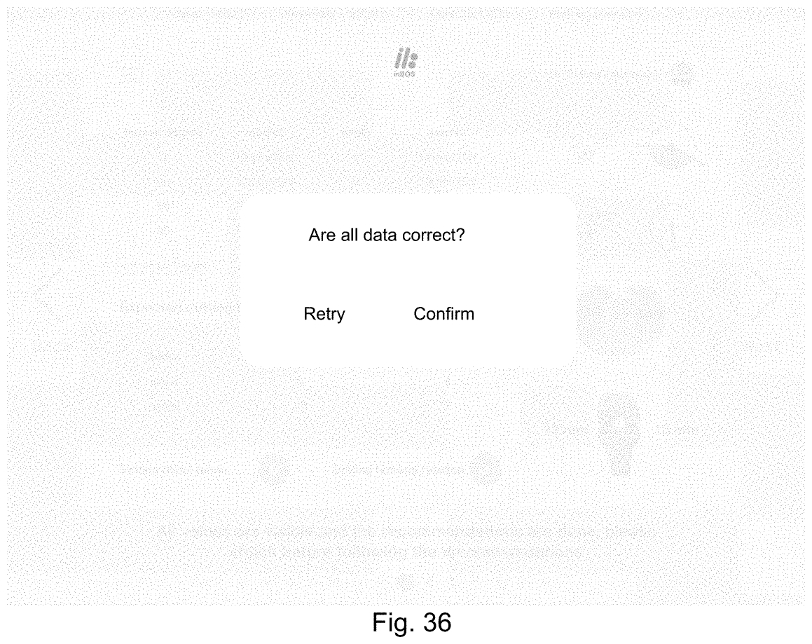

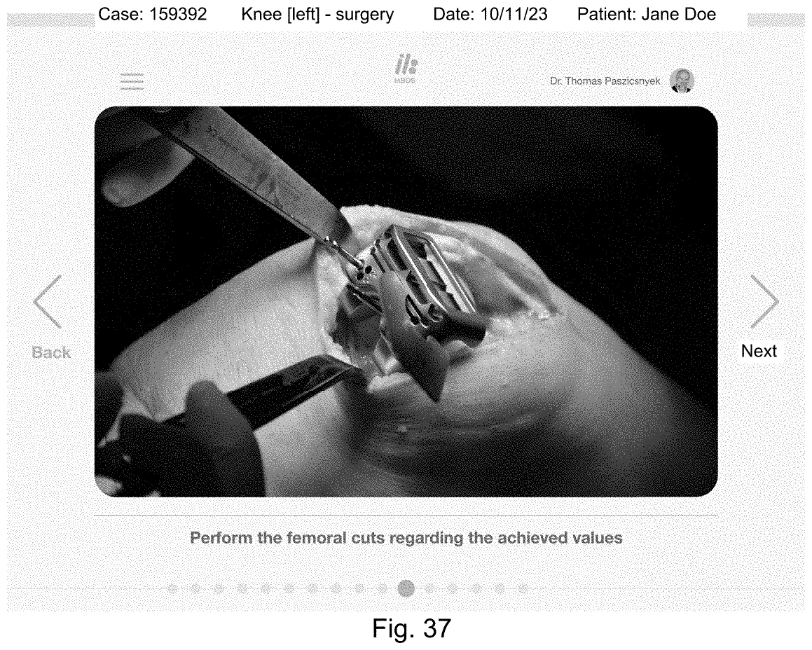

Based on the reference measurements, a recommendation may be generated (FIG. 35). The recommendation may include an expected cutting thickness 4800 for the femur as well as distal femur angle 4802 and femoral rotation 4804. The client device may prompt the user to confirm that data/recommendations are correct retry measurements (FIG. 36). Femoral cuts may be performed to match the recommendations (FIG. 37).

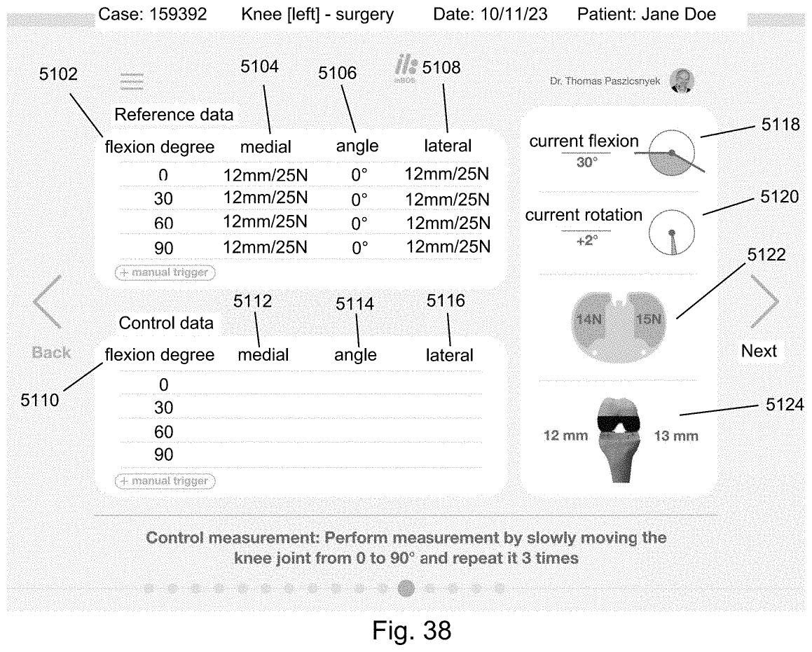

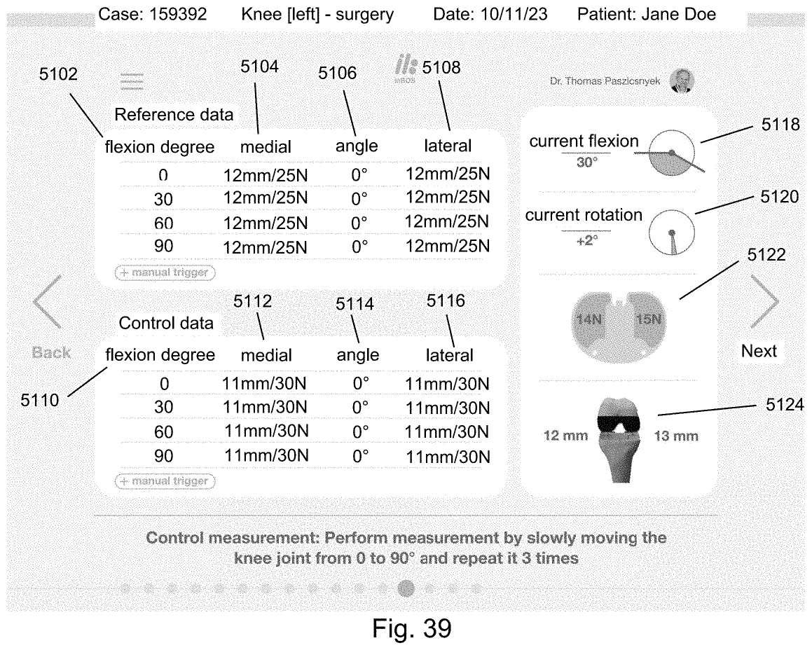

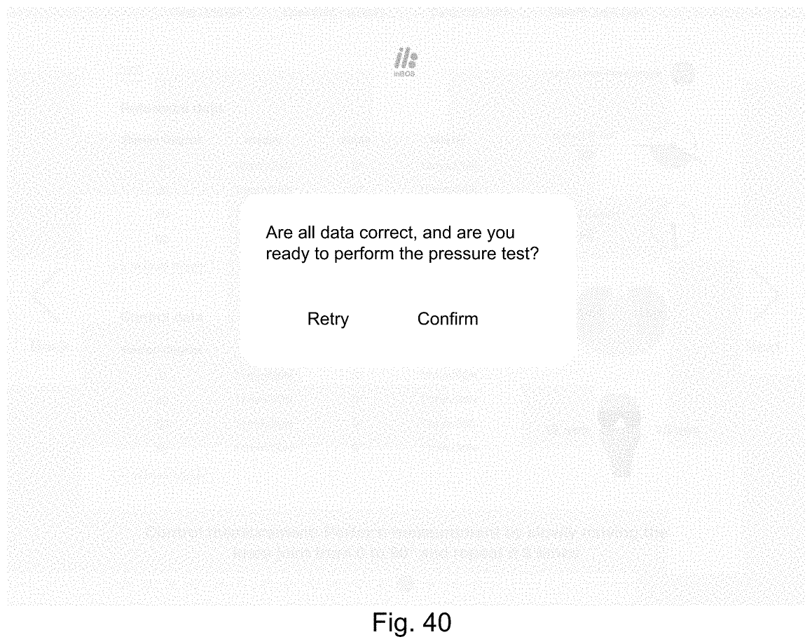

FIGS. 38 and 39 present exemplary interfaces for performing control measurements according to an embodiment of the present invention. Control measurements may be performed after the femoral cuts. In the illustrated embodiment, the client device instructs the user to move the knee joint from 0° to 90° and repeat the procedure an additional three times. The previously measured reference data including flexion degree 5102, paddle distance/force at the medial collateral ligament 5104 and the lateral collateral ligament 5108, and intercondylar angle 5106 may be provided for comparison with the control measurements. Control measurement data from the functional measurement device may be recorded during movement of the knee joint and rendered on the client device. The control measurements include flexion degree 5110, paddle distance/force at the medial collateral ligament 5112 and the lateral collateral ligament 5116, and intercondylar angle 5114. The client device may provide the user an option to proceed by confirming that the measurement data is correct to proceed to pressure testing or retry performing control measurements (FIG. 40).

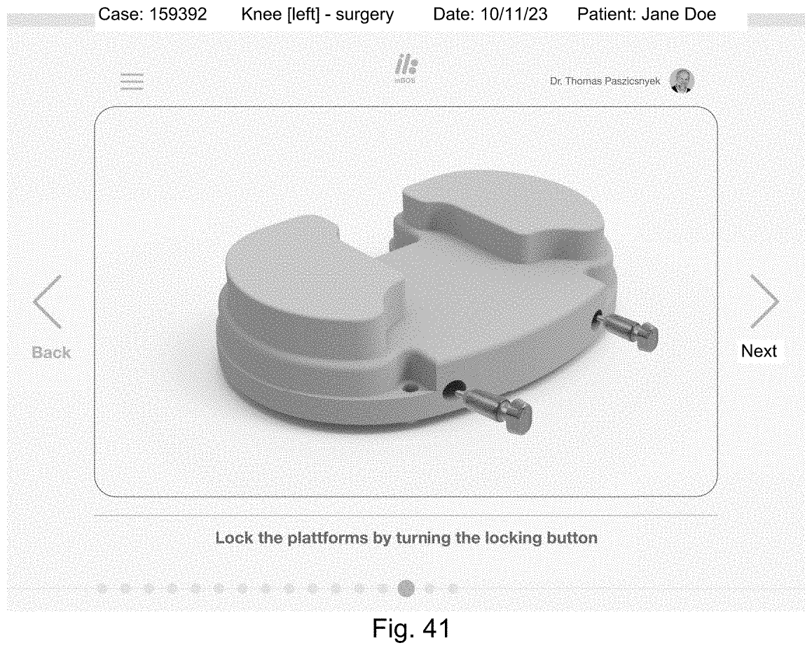

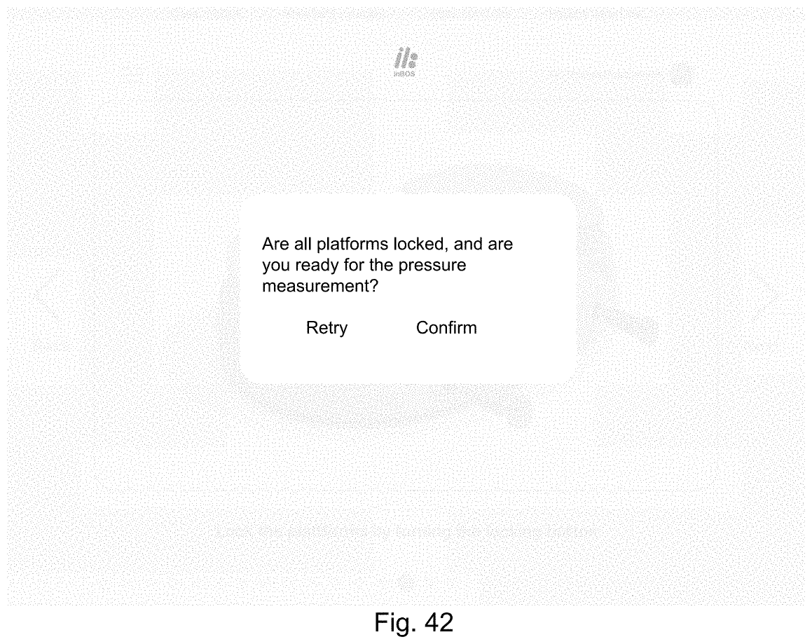

FIG. 41 presents a client device interface providing instructions for locking a functional measurement device by turning locking buttons for each paddle according to an embodiment of the present invention. The client device may confirm that the paddles are locked and provide option to retry locking or confirm and proceed with pressure measurement (FIG. 42).

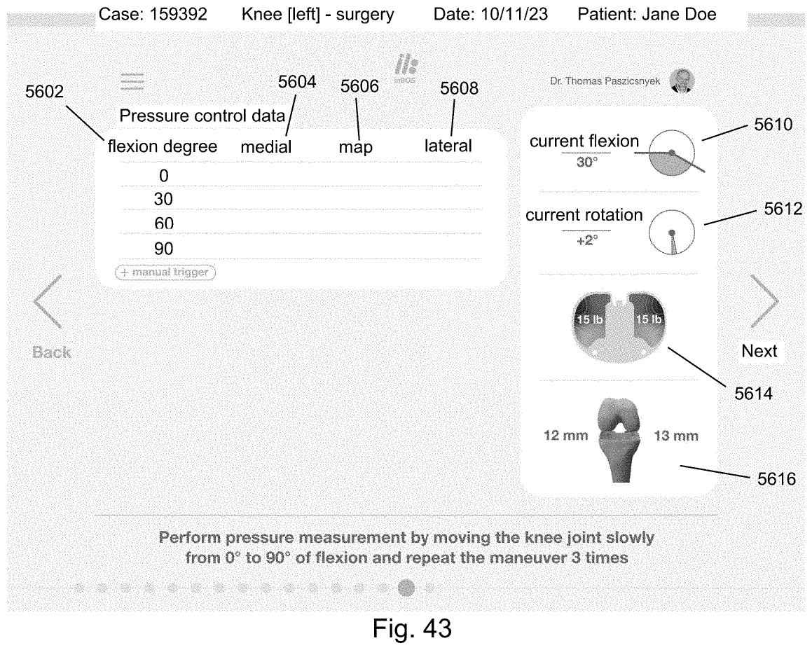

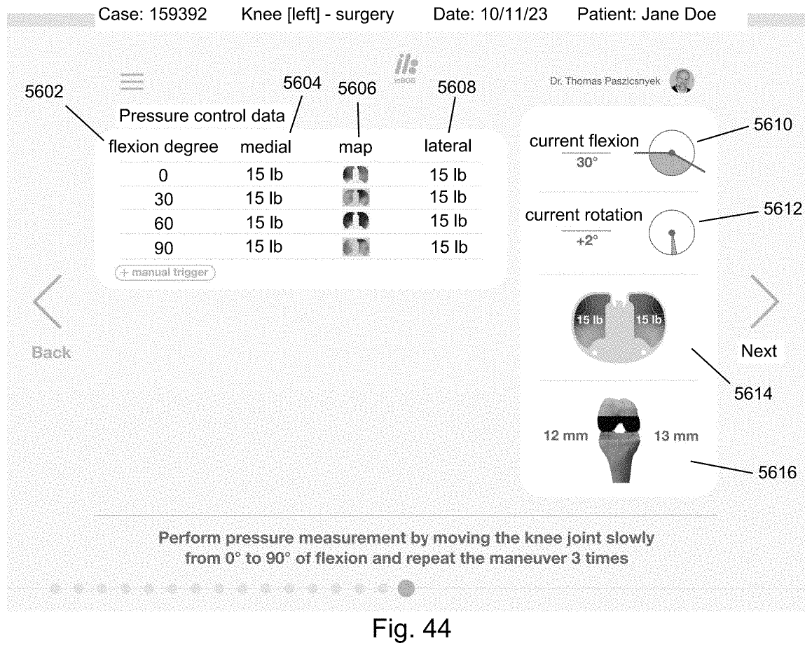

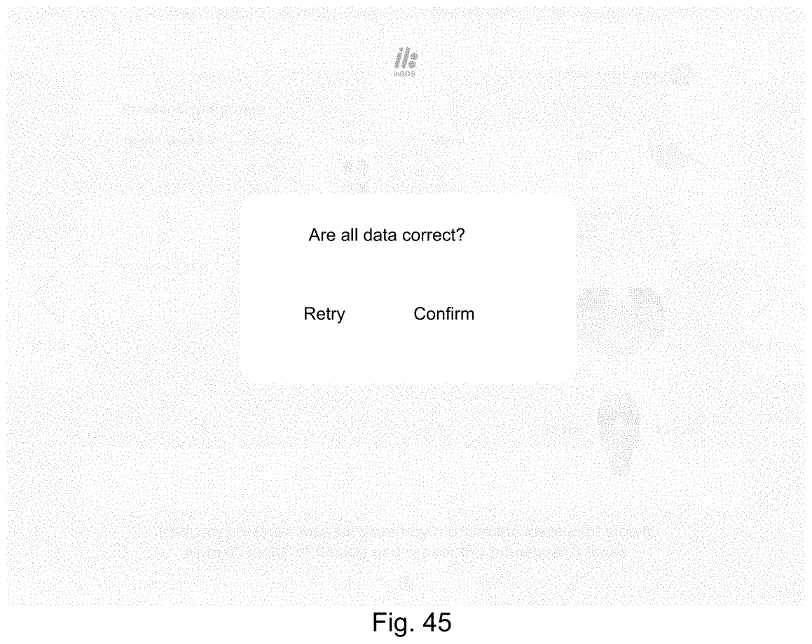

FIGS. 43 and 44 present exemplary interfaces for performing pressure measurements according to an embodiment of the present invention. In the illustrated embodiment, the client device instructs the user to move the knee joint from 0° to 90° and repeat the procedure an additional three times. Pressure data from the functional measurement device may be recorded during movement of the knee joint and rendered on the client device showing flexion degree 5602, paddle load at the medial collateral ligament 5604, paddle load on the lateral collateral ligament 5608, a load distribution map 5606, current flexion 5610, current rotation 5612, functional measurement device paddle loads 5614, and functional measurement device paddle distances 5616. The client device may provide the user an option to proceed by confirming that the pressure measurement data is correct or retry performing pressure measurements (FIG. 45).

FIGS. 1 through 45 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

It should be understood that various aspects of the embodiments of the present invention could be implemented in hardware, firmware, software, or combinations thereof. In such embodiments, the various components and/or steps would be implemented in hardware, firmware, and/or software to perform the functions of the present invention. That is, the same piece of hardware, firmware, or module of software could perform one or more of the illustrated blocks (e.g., components or steps). In software implementations, computer software (e.g., programs or other instructions) and/or data is stored on a machine-readable medium as part of a computer program product and is loaded into a computer system or other device or machine via a removable storage drive, hard drive, or communications interface. Computer programs (also called computer control logic or computer-readable program code) are stored in a main and/or secondary memory, and executed by one or more processors (controllers, or the like) to cause the one or more processors to perform the functions of the invention as described herein. In this document, the terms “machine readable medium,” “computer-readable medium,” “computer program medium,” and “computer usable medium” are used to generally refer to media such as a random access memory (RAM); a read only memory (ROM); a removable storage unit (e.g., a magnetic or optical disc, flash memory device, or the like); a hard disk; or the like.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

What is claimed is:

1. A method, in a data processing system comprising a processor and a memory, for guiding knee replacement procedures, the method comprising:
receiving, by a client device, first data values from one or more sensors and one or more functional measurement devices;
performing, by the client device, a reference measurement based on the first data values from the one or more sensors and the one or more functional measurement devices prior to a femoral cut;
rendering the reference measurement on the client device;
generating joint replacement recommendations based on the reference measurement;
receiving second data values from the one or more sensors and the one or more functional measurement devices;
performing a control measurement based on the second data values from the one or more sensors and the one or more functional measurement devices after a femoral cut based on the joint replacement recommendations;
rendering the control measurement on the client device;
receiving third data values from the one or more functional measurement devices;
performing a pressure test that measures loading, balance, and kinematic behavior at medial and lateral condyles based on the third data values; and
rendering the pressure test on the client device.

2. The method of claim 1 further comprising the client device detecting a pairing signal broadcasted from the one or more sensors and establishing a connection with the one or more sensors.

3. The method of claim 1 further comprising calibrating the one or more sensors based on movement of a joint associated with the one or more sensors, the movement comprising at least one of moving forward and backward, flexing and extending the joint attached to the one or more sensors, and rotating the joint.

4. The method of claim 1 further comprising the client device detecting a pairing signal broadcasted from the one or more functional measurement devices and establishing a connection with the one or more sensors.

5. The method of claim 1 wherein the one or more functional measurement devices are switched on by physically depressing and holding one or more paddles on the functional measurement device.

6. The method of claim 5 wherein calibrating the one or more functional measurement devices further comprises releasing the one or more paddles after the depressing.

7. The method of claim 1 wherein the one or more functional measurement devices include one or more paddles that measure distance traveled by the paddle surfaces when compressed between a tibial surface and the medial and lateral condyles.

8. The method of claim 1 wherein performing the reference measurement further comprises recording reference data values from the one or more sensors and the functional measurement devices at given angles.

9. The method of claim 8 wherein the reference data values include paddle heights at the medial and lateral condyles along with corresponding pressures.

10. The method of claim 1 wherein the joint replacement recommendations include determining and/or calculating one or more of ideal joint planes, alignment, cutting planes, cutting thickness, distal femur angle, femoral rotation, implant types and sizes.

11. The method of claim 1 wherein performing the control measurement further comprises recording control data values from the one or more sensors and the functional measurement devices at given angles.

12. The method of claim 11 wherein the control data values include paddle heights at the medial and lateral condyles along with corresponding pressures.

13. A system for guiding knee replacement procedures, the system comprising:
a processor; and
a memory having executable instructions stored thereon that when executed by the processor cause the processor to:
receive first data values from one or more sensors and one or more functional measurement devices;
perform a reference measurement based on the first data values from the one or more sensors and the one or more functional measurement devices prior to a femoral cut;
render the reference measurement on a user interface;
generate joint replacement recommendations based on the reference measurement;
receive second data values from the one or more sensors and the one or more functional measurement devices;
perform a control measurement based on the second data values from the one or more sensors and the one or more functional measurement devices after a femoral cut based on the joint replacement recommendations;
render the control measurement on the user interface;
receive third data values from the one or more functional measurement devices;
perform a pressure test that measures loading, balance, and kinematic behavior at the medial and lateral condyles based on the third data values; and
render the pressure test on the user interface.

14. The system of claim 13 further comprising the processor configured to:
detect a pairing signal broadcasted from the one or more sensors; and
establish a connection with the one or more sensors.

15. The system of claim 13 further comprising the processor configured to calibrate the one or more sensors based on movement of a joint associated with the one or more sensors, the movement comprising at least one of moving forward and backward, flexing and extending the joint attached to the one or more sensors, and rotating the joint.

16. The system of claim 13 further comprising the processor configured to:
detect a pairing signal broadcasted from the one or more functional measurement devices; and
establish a connection with the one or more sensors.

17. The system of claim 13 wherein the one or more functional measurement devices are switched on by physically depressing and holding one or more paddles on the functional measurement device.

18. The system of claim 17 wherein the one or more functional measurement devices are calibrated by releasing the one or more paddles after the depressing.

19. The system of claim 13 wherein the one or more functional measurement devices include one or more paddles that measure distance traveled by the paddle surfaces when compressed between a tibial surface and the medial and lateral condyles.

20. The system of claim 19 wherein the joint replacement recommendations include determining and/or calculating one or more of ideal joint planes, alignment, cutting planes, cutting thickness, distal femur angle, femoral rotation, implant types and sizes.
